SEQUENCE LISTING (42 sequences)

SEQ ID NO: 1 - DNA, 30 bp, Artificial Sequence
  Description of Artificial SequenceDesigned primer based on nucleotide sequences of human H-Ras and restriction site for XhoI
ctcgagatgacggaatataagctggtggtg

SEQ ID NO: 2 - DNA, 60 bp, Artificial Sequence
  Description of Artificial SequenceDesigned primer based on nucleotide sequences of human c-Raf1 and human H-Ras
agtgttgcttgtcttagaaggggtaccacctccggagccgttcagcttccgcagcttgtg

SEQ ID NO: 3 - DNA, 27 bp, Artificial Sequence
  Description of Artificial SequenceDesigned primer based on nucleotide sequences of human c-Raf1 and restriction site for KpnI
ggtaccccttctaagacaagcaacact

SEQ ID NO: 4 - DNA, 30 bp, Artificial Sequence
  Description of Artificial SequenceDesigned primer based on nucleotide sequences of human c-Raf1 and restriction site for NotI
gcggccgcccaggaaatctacttgaagttc

SEQ ID NO: 5 - DNA, 24 bp, Artificial Sequence
  Description of Artificial SequenceDesigned primer based on nucleotide sequence of multiple cloning site of pBluescript-SKII(+)
cgccagggttttcccagtcacgac

SEQ ID NO: 6 - DNA, 27 bp, Artificial Sequence
  Description of Artificial SequenceDesigned primer based on nucleotide sequence of multiple cloning site of pBluescript-SKII(+)
agcggataacaatttcacacaggaaac

SEQ ID NO: 7 - DNA, 30 bp, Artificial Sequence
  Description of Artificial SequenceDesigned primer based on nucleotide sequences of aequorea EYFP and restriction site for BamHI
ggatccggcatggtgagcaagggcgaggag

SEQ ID NO: 8 - DNA, 36 bp, Artificial Sequence
  Description of Artificial SequenceDesigned primer based on nucleotide sequences of aequorea ECFP and restriction sites for BamHI, KpnI and XhoI
ggatccggtacctcgagcttgtacagctcgtccatg

SEQ ID NO: 9 - DNA, 30 bp, Artificial Sequence
  Description of Artificial SequenceDesigned primer based on nucleotide sequences of aequorea ECFP and restriction site for NotI
gcggccgcatggtgagcaagggcgaggagc

SEQ ID NO: 10 - DNA, 27 bp, Artificial Sequence
  Description of Artificial SequenceDesigned primer based on nucleotide sequences of aequorea ECFP and restriction site for BglII
agatctacagctcgtccatgccgagag

SEQ ID NO: 11 - DNA, 2223 bp, Artificial Sequence
  Description of Artificial SequenceDesigned plasmid based on nucleotide sequences of human H-Ras, human c-Raf1, aequorea EYFP and aequorea ECFP
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgaagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagctcgagatgacggaatataagctggtggtggtgggcgccggcggtgtgggcaagagtgcgctgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaacggctccggaggtggtaccccttctaagacaagcaacactatccgtgttttcttgccgaacaagcaaagaacagtggtcaatgtgcgaaatggaatgagcttgcatgactgccttatgaaagcactcaaggtgaggggcctgcaaccagagagctgtgcagtgttcagacttctccacgaacacaaaggtaaaaaagcacgcttagattggaatactgaagctgcgtctttgattggagaagaacttcacgtagatttcctgggcggccgcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacttgagcacccagtccgccctgagcaaagaccccaacgagaagcgcgatcacatggtcctgctggagttcgtgaccgccgccgggatcactctcggcatggacgagctgtag

SEQ ID NO: 12 - protein, 740 aa, Artificial Sequence
  Description of Artificial SequenceDesigned plasmid based on nucleotide sequences of human H-Ras, human c-Raf1, aequorea EYFP and aequorea ECFP
MVSKGEELFTGVVPILVELDGDVNGHKFSVSGEGEGDATYGKLTLKFICTTGKLPVPWPTLVTTFGYGLKCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYKLEMTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNGSGGGTPSKTSNTIRVFLPNKQRTVVNVRNGMSLHDCLMKALKVRGLQPESCAVFRLLHEHKGKKARLDWNTEAASLIGEELHVDFLGGRMVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGITLGMDEL

SEQ ID NO: 13 - DNA, 30 bp, Artificial Sequence
  Description of Artificial SequenceDesigned primer based on nucleotide sequences of human Rap1A and restriction site for XhoI
ggctcgagatgcgtgagtacaagctagtgg

SEQ ID NO: 14 - DNA, 92 bp, Artificial Sequence
  Description of Artificial SequenceDesigned primer based on nucleotide sequences of human RalGDS and human Rap1A
gcggatgatacagcagtcgccacctccggatccgccggtacctccaccaccggttccacctccggagccattgatctttgactttgcagaag

SEQ ID NO: 15 - DNA, 21 bp, Artificial Sequence
  Description of Artificial SequenceDesigned primer based on nucleotide sequence of human RalGDS
ggcgactgctgtatcatccgc

SEQ ID NO: 16 - DNA, 31 bp, Artificial Sequence
  Description of Artificial SequenceDesigned primer based on nucleotide sequences of human RalGDS and restriction site for NotI
cgcggccgccccgcttcttgaggacaaagtc

SEQ ID NO: 17 - DNA, 41 bp, Artificial Sequence
  Description of Artificial SequenceDesigned primer based on nucleotide sequences of aequorea ECFP and restriction sites for BamHI, KpnI and XhoI
ggatccggtacctcgagggcggcggtcacgaactccagcag

SEQ ID NO: 18 - DNA, 2238 bp, Artificial Sequence
  Description of Artificial SequenceDesigned plasmid based on nucleotide sequences of human Rap1A, human RalGDS, aequorea EYFP and aequorea ECFP
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgaagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccctcgagatgcgtgagtacaagctagtggtccttggttcaggaggcgttgggaagtctgctctgacagttcagtttgttcagggaatttttgttgaaaaatatgacccaacgatagaagattcctacagaaagcaagttgaagtcgattgccaacagtgtatgctcgaaatcctggatactgcagggacagagcaatttacagcaatgagggatttgtatatgaagaacggccaaggttttgcactagtatattctattacagctcagtccacgtttaacgacttacaggacctgagggaacagattttacgggttaaggacacggaagatgttccaatgattttggttggcaataaatgtgacctggaagatgagcgagtagttggcaaagagcagggccagaatttagcaagacagtggtgtaactgtgcctttttagaatcttctgcaaagtcaaagatcaatgttaatgagatattttatgacctggtcagacagataaataggaaaacaccagtggaaggctccggaggtggaaccggtggtggaggtaccggcggatccggaggtggcgactgctgtatcatccgcgtcagcctggacgtggacaatggcaacatgtacaagagcatcctggtgaccagccaagataaggctccggctgtaatccgcaaggccatggacaaacacaacctggaggaggaggagccggaggactatgagctgctgcagattctctcagatgaccggaagctgaagatccctgaaaacgccaacgtcttctatgccatgaactctaccgccaactatgactttgtcctcaagaagcggggcggccgcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacttgagcacccagtccgccctgagcaaagaccccaacgagaagcgcgatcacatggtcctgctggagttcgtgaccgccgccgggatcactctcggcatggacgagctgtag

SEQ ID NO: 19 - protein, 745 aa, Artificial Sequence
  Description of Artificial SequenceDesigned plasmid based on nucleotide sequences of human Rap1A, human RalGDS, aequorea EYFP and aequorea ECFP
MVSKGEELFTGVVPILVELDGDVNGHKFSVSGEGEGDATYGKLTLKFICTTGKLPVPWPTLVTTFGYGLKCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAALEMREYKLVVLGSGGVGKSALTVQFVQGIFVEKYDPTIEDSYRKQVEVDCQQCMLEILDTAGTEQFTAMRDLYMKNGQGFALVYSITAQSTFNDLQDLREQILRVKDTEDVPMILVGNKCDLEDERVVGKEQGQNLARQWCNCAFLESSAKSKINVNEIFYDLVRQINRKTPVEGSGGGTGGGGTGGSGGGDCCIIRVSLDVDNGNMYKSILVTSQDKAPAVIRKAMDKHNLEEEEPEDYELLQILSDDRKLKIPENANVFYAMNSTANYDFVLKKRGGRMVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGITLGMDEL

SEQ ID NO: 20 - DNA, 27 bp, Artificial Sequence
  Description of Artificial SequenceDesigned primer based on nucleotide sequences of human R-Ras and restriction site for XhoI
cccctcgagacacacaagctggtggtc

SEQ ID NO: 21 - DNA, 33 bp, Artificial Sequence
  Description of Artificial SequenceDesigned primer based on nucleotide sequences of human R-Ras and restriction site for KpnI
gccggtaccgccactgggagggctcggtgggag

SEQ ID NO: 22 - DNA, 2223 bp, Artificial Sequence
  Description of Artificial SequenceDesigned plasmid based on nucleotide sequences of human R-Ras, human c-Raf1, aequorea EYFP and aequorea ECFP
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgaagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagctcgagacacacaagctggtggtcgtgggcggcggcggcgtgggcaagagcgcgctgaccatccagttcatccagtcctacttcgtgtctgactacgaccccactattgaggactcctacacgaagatctgcagtgtggatggcatcccagcccggctggacatcctggacaccgcgggccaggaagagttcggggccatgagagagcagtacatgcgtgctggccacggcttcctgctggtgttcgccattaatgaccggcagagtttcaacgaggtgggcaagctcttcacgcagattctgcgggtcaaggaccgcgacgacttccccgttgtgttggtcgggaacaaggcagatctggagtcacagcgccaggtcccccgatcagaagcctctgccttcggcgcctcccaccacgtggcctactttgaggcctcggccaaactgcgtctcaacgtggacgaggcttttgagcagctggtgcgggctgtccggaaataccaggaacaagagctcccaccgagccctcccagtggtaccccttctaagacaagcaacactatccgtgttttcttgccgaacaagcaaagaacagtggtcaatgtgcgaaatggaatgagcttgcatgactgccttatgaaagcactcaaggtgaggggcctgcaaccagagagctgtgcagtgttcagacttctccacgaacacaaaggtaaaaaagcacgcttagattggaatactgaagctgcgtctttgattggagaagaacttcacgtagatttcctgggcggccgcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacttgagcacccagtccgccctgagcaaagaccccaacgagaagcgcgatcacatggtcctgctggagttcgtgaccgccgccgggatcactctcggcatggacgagctgtag

SEQ ID NO: 23 - protein, 740 aa, Artificial Sequence
  Description of Artificial SequenceDesigned plasmid based on nucleotide sequences of human R-Ras, human c-Raf1, aequorea EYFP and aequorea ECFP
MVSKGEELFTGVVPILVELDGDVNGHKFSVSGEGEGDATYGKLTLKFICTTGKLPVPWPTLVTTFGYGLKCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYKLETHKLVVVGGGGVGKSALTIQFIQSYFVSDYDPTIEDSYTKICSVDGIPARLDILDTAGQEEFGAMREQYMRAGHGFLLVFAINDRQSFNEVGKLFTQILRVKDRDDFPVVLVGNKADLESQRQVPRSEASAFGASHHVAYFEASAKLRLNVDEAFEQLVRAVRKYQEQELPPSPPSGTPSKTSNTIRVFLPNKQRTVVNVRNGMSLHDCLMKALKVRGLQPESCAVFRLLHEHKGKKARLDWNTEAASLIGEELHVDFLGGRMVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGITLGMDEL

SEQ ID NO: 24 - DNA, 22 bp, Artificial Sequence
  Description of Artificial SequenceDesigned primer based on nucleotide sequence of human H-Ras
ggaatcctctagagtggggtcg

SEQ ID NO: 25 - DNA, 22 bp, Artificial Sequence
  Description of Artificial SequenceDesigned primer based on nucleotide sequence of human H-Ras
cgaccccactctagaggattcc

SEQ ID NO: 26 - DNA, 2223 bp, Artificial Sequence
  Description of Artificial SequenceDesigned plasmid based on nucleotide sequences of human H-Ras, human c-Raf1, aequorea EYFP and aequorea ECFP
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgaagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagctcgagatgacggaatataagctggtggtggtgggcgccggcggtgtgggcaagagtgcgctgaccatccagctgatccagaaccattttgtggacgaatacgaccccactctagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaacggctccggaggtggtaccccttctaagacaagcaacactatccgtgttttcttgccgaacaagcaaagaacagtggtcaatgtgcgaaatggaatgagcttgcatgactgccttatgaaagcactcaaggtgaggggcctgcaaccagagagctgtgcagtgttcagacttctccacgaacacaaaggtaaaaaagcacgcttagattggaatactgaagctgcgtctttgattggagaagaacttcacgtagatttcctgggcggccgcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacttgagcacccagtccgccctgagcaaagaccccaacgagaagcgcgatcacatggtcctgctggagttcgtgaccgccgccgggatcactctcggcatggacgagctgtag

SEQ ID NO: 27 - protein, 740 aa, Artificial Sequence
  Description of Artificial SequenceDesigned plasmid based on nucleotide sequences of human H-Ras, human c-Raf1, aequorea EYFP and aequorea ECFP
MVSKGEELFTGVVPILVELDGDVNGHKFSVSGEGEGDATYGKLTLKFICTTGKLPVPWPTLVTTFGYGLKCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYKLEMTEYKLVVVGAGGVGKSALTIQLIQNHFVDEYDPTLEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNGSGGGTPSKTSNTIRVFLPNKQRTVVNVRNGMSLHDCLMKALKVRGLQPESCAVFRLLHEHKGKKARLDWNTEAASLIGEELHVDFLGGRMVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGITLGMDEL

SEQ ID NO: 28 - DNA, 27 bp, Artificial Sequence
  Description of Artificial SequenceDesigned primer based on nucleotide sequence of human c-Raf1
ctcgagccttctaagacaagcaacact

SEQ ID NO: 29 - DNA, 22 bp, Artificial Sequence
  Description of Artificial SequenceDesigned primer based on nucleotide sequence of aequorea ECFP
cgtcgccgtccagctcgaccag

SEQ ID NO: 30 - DNA, 2205 bp, Artificial Sequence
  Description of Artificial SequenceDesigned plasmid based on nucleotide sequences of human Rap1A, human c-Raf1, aequorea EYFP and aequorea ECFP
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccctcgagatgcgtgagtacaagctagtggtccttggttcaggaggcgttgggaagtctgctctgacagttcagtttgttcagggaatttttgttgaaaaatatgacccaacgatagaagattcctacagaaagcaagttgaagtcgattgccaacagtgtatgctcgaaatcctggatactgcagggacagagcaatttacagcaatgagggatttgtatatgaagaacggccaaggttttgcactagtatattctattacagctcagtccacgtttaacgacttacaggacctgagggaacagattttacgggttaaggacacggaagatgttccaatgattttggttggcaataaatgtgacctggaagatgagcgagtagttggcaaagagcagggccagaatttagcaagacagtggtgtaactgtgcctttttagaatcttctgcaaagtcaaagatcaatgttaatgagatattttatgacctggtcagacagataaataggaaaacaccagtggaaggctccggaggtggaaccggtggtggaggtaccccttctaagacaagcaacactatccgtgttttcttgccgaacaagcaaagaacagtggtcaatgtgcgaaatggaatgagcttgcatgactgccttatgaaagcactcaaggtgaggggcctgcaaccagagagctgtgcagtgttcagacttctccacgaacacaaaggtaaaaaagcacgcttagattggaatactgaagctgcgtctttgattggagaagaacttcacgtagatttcctgggcggccgcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacttgagcacccagtccgccctgagcaaagaccccaacgagaagcgcgatcacatggtcctgctggagttcgtgaccgccgccgggatcactctcggcatggacgagctgtag

SEQ ID NO: 31 - protein, 734 aa, Artificial Sequence
  Description of Artificial SequenceDesigned plasmid based on nucleotide sequences of human Rap1A, human c-Raf1, aequorea EYFP and aequorea ECFP
MVSKGEELFTGVVPILVELDGDVNGHKFSVSGEGEGDATYGKLTLKFICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSALSKDPNEKRDHMVLLEFVTAALEMREYKLVVLGSGGVGKSALTVQFVQGIFVEKYDPTIEDSYRKQVEVDCQQCMLEILDTAGTEQFTAMRDLYMKNGQGFALVYSITAQSTFNDLQDLREQILRVKDTEDVPMILVGNKCDLEDERVVGKEQGQNLARQWCNCAFLESSAKSKINVNEIFYDLVRQINRKTPVEGSGGGTGGGGTPSKTSNTIRVFLPNKQRTVVNVRNGMSLHDCLMKALKVRGLQPESCAVFRLLHEHKGKKARLDWNTEAASLIGEELHVDFLGGRMVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGITLGMDEL

SEQ ID NO: 32 - DNA, 38 bp, Artificial Sequence
  Description of Artificial SequenceDesigned primer based on nucleotide sequence of aequorea ECFP
tctagagcggcccagctcgtccatgccgagagtgatcc

SEQ ID NO: 33 - DNA, 2295 bp, Artificial Sequence
  Description of Artificial SequenceDesigned plasmid based on nucleotide sequences of human H-Ras, human c-Raf1, aequorea EYFP, aequorea ECFP and human K-Ras
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgaagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagctcgagatgacggaatataagctggtggtggtgggcgccggcggtgtgggcaagagtgcgctgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaacggctccggaggtggtaccccttctaagacaagcaacactatccgtgttttcttgccgaacaagcaaagaacagtggtcaatgtgcgaaatggaatgagcttgcatgactgccttatgaaagcactcaaggtgaggggcctgcaaccagagagctgtgcagtgttcagacttctccacgaacacaaaggtaaaaaagcacgcttagattggaatactgaagctgcgtctttgattggagaagaacttcacgtagatttcctgggcggccgcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacttgagcacccagtccgccctgagcaaagaccccaacgagaagcgcgatcacatggtcctgctggagttcgtgaccgccgccgggatcactctcggcatggacgagctgggccgctctagaaagatgagcaaagatggtaaaaagaagaaaaagaagtcaaagacaaagtgtgtaattatgtaa

SEQ ID NO: 34 - protein, 764 aa, Artificial Sequence
  Description of Artificial SequenceDesigned plasmid based on nucleotide sequences of human H-Ras, human c-Raf1, aequorea EYFP, aequorea ECFP and human K-Ras
MVSKGEELFTGVVPILVELDGDVNGHKFSVSGEGEGDATYGKLTLKFICTTGKLPVPWPTLVTTFGYGLKCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYKLEMTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNGSGGGTPSKTSNTIRVFLPNKQRTVVNVRNGMSLHDCLMKALKVRGLQPESCAVFRLLHEHKGKKARLDWNTEAASLIGEELHVDFLGGRMVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGITLGMDELGRSRKMSKDGKKKKKKSKTKCVIM

SEQ ID NO: 35 - DNA, 2277 bp, Artificial Sequence
  Description of Artificial SequenceDesigned plasmid based on nucleotide sequences of human Rap1A, human c-Raf1, aequorea EYFP, aequorea ECFP and human K-Ras
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccctcgagatgcgtgagtacaagctagtggtccttggttcaggaggcgttgggaagtctgctctgacagttcagtttgttcagggaatttttgttgaaaaatatgacccaacgatagaagattcctacagaaagcaagttgaagtcgattgccaacagtgtatgctcgaaatcctggatactgcagggacagagcaatttacagcaatgagggatttgtatatgaagaacggccaaggttttgcactagtatattctattacagctcagtccacgtttaacgacttacaggacctgagggaacagattttacgggttaaggacacggaagatgttccaatgattttggttggcaataaatgtgacctggaagatgagcgagtagttggcaaagagcagggccagaatttagcaagacagtggtgtaactgtgcctttttagaatcttctgcaaagtcaaagatcaatgttaatgagatattttatgacctggtcagacagataaataggaaaacaccagtggaaggctccggaggtggaaccggtggtggaggtaccccttctaagacaagcaacactatccgtgttttcttgccgaacaagcaaagaacagtggtcaatgtgcgaaatggaatgagcttgcatgactgccttatgaaagcactcaaggtgaggggcctgcaaccagagagctgtgcagtgttcagacttctccacgaacacaaaggtaaaaaagcacgcttagattggaatactgaagctgcgtctttgattggagaagaacttcacgtagatttcctgggcggccgcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacttgagcacccagtccgccctgagcaaagaccccaacgagaagcgcgatcacatggtcctgctggagttcgtgaccgccgccgggatcactctcggcatggacgagctgggccgctctagaaagatgagcaaagatggtaaaaagaagaaaaagaagtcaaagacaaagtgtgtaattatgtaa

SEQ ID NO: 36 - protein, 758 aa, Artificial Sequence [sequence omitted]

SEQ ID NO: 37 - DNA, 2292 bp, Artificial Sequence
  Description of Artificial SequenceDesigned plasmid based on nucleotide sequences of human Rac1, human Pak1, aequorea EYFP, aequorea ECFP and human K-Ras
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccctcgagaaagagaaagagcggccagagatttctctcccttcagattttgaacacacaattcatgtcggttttgatgctgtcacaggggagtttacgggaatgccagagcagtgggcccgcttgcttcagacatcaaatatcactaagtcggagcagaagaaaaacccgcaggctgttctggatgtgttggagttttacaactcgaagaagacatccaacagccagaaatacatgagctttacagataagtcagcttccggaggtggaaccggtggtggaggtaccatgcaggccatcaagtgtgtggtggtgggagacggagctgtaggtaaaacttgcctactgatcagttacacaaccaatgcatttcctggagaatatatccctactgtctttgacaattattctgccaatgttatggtagatggaaaaccggtgaatctgggcttatgggatacagctggacaagaagattatgacagattacgccccctatcctatccgcaaacagatgtgttcttaatttgcttttcccttgtgagtcctgcatcatttgaaaatgtccgtgcaaagtggtatcctgaggtgcggcaccactgtcccaacactcccatcatcctagtgggaactaaacttgatcttagggatgataaagacacgatcgagaaactgaaggagaagaagctgactcccatcacctatccgcagggtctagccatggctaaggagattggtgctgtaaaatacctggagtgctcggcgctcacacagcgaggcctcaagacagtgtttgacgaagcgatccgagcagtccgcggccgcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacttgagcacccagtccgccctgagcaaagaccccaacgagaagcgcgatcacatggtcctgctggagttcgtgaccgccgccgggatcactctcggcatggacgagctgggccgctctagaaagatgagcaaagatggtaaaaagaagaaaaagaagtcaaagacaaagtgtgtaattatgtaa

SEQ ID NO: 38 - protein, 763 aa, Artificial Sequence
  Description of Artificial SequenceDesigned plasmid based on nucleotide sequences of human Rac1, human Pak1, aequorea EYFP, aequorea ECFP and human K-Ras
MVSKGEELFTGVVPILVELDGDVNGHKFSVSGEGEGDATYGKLTLKFICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSALSKDPNEKRDHMVLLEFVTAALEKEKERPEISLPSDFEHTIHVGFDAVTGEFTGMPEQWARLLQTSNITKSEQKKNPQAVLDVLEFYNSKKTSNSQKYMSFTDKSASGGGTGGGGTMQAIKCVVVGDGAVGKTCLLISYTTNAFPGEYIPTVFDNYSANVMVDGKPVNLGLWDTAGQEDYDRLRPLSYPQTDVFLICFSLVSPASFENVRAKWYPEVRHHCPNTPIILVGTKLDLRDDKDTIEKLKEKKLTPITYPQGLAMAKEIGAVKYLECSALTQRGLKTVFDEAIRAVRGRMVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGITLGMDELGRSRKMSKDGKKKKKKSKTKCVIM

SEQ ID NO: 39 - DNA, 2289 bp, Artificial Sequence
  Description of Artificial SequenceDesigned plasmid based on nucleotide sequences of human Cdc42, human Pak1, aequorea EYFP, aequorea ECFP and human K-Ras
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccctcgagaaagagaaagagcggccagagatttctctcccttcagattttgaacacacaattcatgtcggttttgatgctgtcacaggggagtttacgggaatgccagagcagtgggcccgcttgcttcagacatcaaatatcactaagtcggagcagaagaaaaacccgcaggctgttctggatgtgttggagttttacaactcgaagaagacatccaacagccagaaatacatgagctttacagataagtcagcttccggaggtggaaccggtggtggaggtacccagacaattaagtgtgttgttgtgggcgatggtgctgttggtaaaacatgtctcctgatatcctacacaacaaacaaatttccatcggaatatgtaccgactgtttttgacaactatgcagtcacagttatgattggtggagaaccatatactcttggactttttgatactgcagggcaagaggattatgacagattacgaccgctgagttatccacaaacagatgtatttctagtctgtttttcagtggtctctccatcttcatttgaaaacgtgaaagaaaagtgggtgcctgagataactcaccactgtccaaagactcctttcttgcttgttgggactcaaattgatctcagagatgacccctctactattgagaaacttgccaagaacaaacagaagcctatcactccagagactgctgaaaagctggcccgtgacctgaaggctgtcaagtatgtggagtgttctgcacttacacagagaggtctgaagaatgtgtttgatgaggctatcctagctgccggcggccgcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacttgagcacccagtccgccctgagcaaagaccccaacgagaagcgcgatcacatggtcctgctggagttcgtgaccgccgccgggatcactctcggcatggacgagctgggccgctctagaaagatgagcaaagatggtaaaaagaagaaaaagaagtcaaagacaaagtgtgtaattatgtaa

SEQ ID NO: 40 - protein, 762 aa, Artificial Sequence
  Description of Artificial SequenceDesigned plasmid based on nucleotide sequences of human Cdc42, human Pak1, aequorea EYFP, aequorea ECFP and human K-Ras
MVSKGEELFTGVVPILVELDGDVNGHKFSVSGEGEGDATYGKLTLKFICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSALSKDPNEKRDHMVLLEFVTAALEKEKERPEISLPSDFEHTIHVGFDAVTGEFTGMPEQWARLLQTSNITKSEQKKNPQAVLDVLEFYNSKKTSNSQKYMSFTDKSASGGGTGGGGTQTIKCVVVGDGAVGKTCLLISYTTNKFPSEYVPTVFDNYAVTVMIGGEPYTLGLFDTAGQEDYDRLRPLSYPQTDVFLVCFSVVSPSSFENVKEKWVPEITHHCPKTPFLLVGTQIDLRDDPSTIEKLAKNKQKPITPETAEKLARDLKAVKYVECSALTQRGLKNVFDEAILAAGGRMVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGITLGMDELGRSRKMSKDGKKKKKKSKTKCVIM

SEQ ID NO: 41 - DNA, 2472 bp, Artificial Sequence
  Description of Artificial SequenceDesigned plasmid based on nucleotide sequences of human RhoA, human mDial, aequorea EYFP, aequorea ECFP and human K-Ras
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccctcgacggtaccgaccccactgctcagtcattgcaggacatctcagacgagcaagttcttgtcctctttgagcagatgctggtggatatgaacctgaatgaggagaagcagcagcctttgcgagagaaggacattgtcatcaagagggagatggtgtcgcaatatctgcacacttccaaggctggcatgaaccagaaagagagctctaggtctgccatgatgtacatccaggagctgaggtcgggcttgcgggatatgcacctgcttagctgccttgagtcccttcgagtctctctcaacaataaccctgtcagttgggtgcagacatttggtgctgagggcctagcctccttattggacatcctcaaacgactccatgatgagaaagaggagacttctggaaactacgccggtggtggaggtaccatggctgccatccggaagaaactggtgattgttggtgatggagcctgtggaaagacatgcttgctcatagtcttcagcaaggaccagttcccagaggtgtatgtgcccacagtgtttgagaactatgtggcagatatcgaggtggatggaaagcaggtagagttggctttgtgggacacagctgggcaggaagattatgatcgcctgaggcccctctcctacccagataccgatgttatactgatgtgtttttccatcgacagccctgatagtttagaaaacatcccagaaaagtggaccccagaagtcaagcatttctgtcccaacgtgcccatcatcctggttgggaataagaaggatcttcggaatgatgagcacacaaggcgggagctagccaagatgaagcaggagccggtgaaacctgaagaaggcagagatatggcaaacaggattggcgcttttgggtacatggagtgttcagcaaagaccaaagatggagtgagagaggtttttgaaatggctacgagagctgctctgcaagctagacgtgggaagaaaaaatctggtggcggccgcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacttgagcacccagtccgccctgagcaaagaccccaacgagaagcgcgatcacatggtcctgctggagttcgtgaccgccgccgggatcactctcggcatggacgagctgggccgctctagaaagatgagcaaagatggtaaaaagaagaaaaagaagtcaaagacaaagtgtgtaattatgtaa

SEQ ID NO: 42 - protein, 823 aa, Artificial Sequence
  Description of Artificial SequenceDesigned plasmid based on nucleotide sequences of human RhoA, human mDial, aequorea EYFP, aequorea ECFP and human K-Ras
MVSKGEELFTGVVPILVELDGDVNGHKFSVSGEGEGDATYGKLTLKFICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSALSKDPNEKRDHMVLLEFVTAALDGTDPTAQSLQDISDEQVLVLFEQMLVDMNLNEEKQQPLREKDIVIKREMVSQYLHTSKAGMNQKESSRSAMMYIQELRSGLRDMHLLSCLESLRVSLNNNPVSWVQTFGAEGLASLLDILKRLHDEKEETSGNYAGGGGTMAAIRKKLVIVGDGACGKTCLLIVFSKDQFPEVYVPTVFENYVADIEVDGKQVELALWDTAGQEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQARRGKKKSGGGRMVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGITLGMDELGRSRKMSKDGKKKKKKSKTKCVIM

(19) United States
(12) Patent Application Publication
(10) Pub. No.: US 2004/0053328 A1
(43) Pub. Date: Mar. 18, 2004

(54) Monitoring proteins for the activities of low-molecular- weight gtp-binding proteins

(76) Inventor: Michiyuki Matsuda, Osaka (JP)

Correspondence Address: ARMSTRONG, KRATZ, QUINTOS, HANSON & BROOKS, LLP, 1725 K STREET, NW, SUITE 1000, WASHINGTON, DC, 20006, US

(21) Appl. No.: 10/344,404

(22) PCT Filed: Aug. 13, 2001
(86) PCT No.: PCT/JP01/06967

(30) Foreign Application Priority Data
Aug. 14, 2000 (JP) 2000-245910
Jan. 31, 2001 (WO) PCT/JP01/00631
May 25, 2001 (WO) PCT/JP01/04421

(51) Int. Cl. G01N 33/53; C07K 16/28

(52) U.S. Cl. 435/7.1; 530/388.22

(57) ABSTRACT

The present invention relates to monitoring proteins for the activity of low-molecular-weight GTP-binding proteins, genes encoding the proteins, expression vectors encoding the genes, cells and transgenic animals carrying the expression vectors, methods for measurement of the activity of low-molecular-weight GTP-binding proteins which use the proteins, and screening methods for the regulatory substances of low-molecular-weight GTP-binding proteins.

### BACKGROUND OF THE INVENTION

1. Field of the Invention

The present invention relates to monitoring proteins for the activity of low-molecular-weight GTP-binding proteins, genes encoding the proteins, expression vectors encoding the genes, cells and transgenic animals carrying the expression vectors, methods for the activity of low-molecular-weight GTP-binding proteins which use the proteins, and screening procedures for the substances which regulate the activity of low-molecular-weight GTP-binding proteins.

2. Description of the Related Art

There are many intracellular signaling molecules. Among them, low-molecular-weight GTP-binding proteins, often called as GTP-binding proteins hereafter, have been extensively studied, because there are many proteins belonging to this group and because they play critical roles as molecular switches of various signal transduction cascades. The low-molecular-weight GTP-binding proteins consist of Ras-family, Rho-family, Rab-family, Ran-family, etc (ref. 1). These low-molecular-weight GTP-binding proteins function as critical molecular switches of cell growth, cytoskeleton, intracellular trafficking, and nuclear transport. The low-molecular-weight GTP-binding proteins cycle between GTP-bound inactive and GTP-bound active forms (FIG. 1). The GTP-bound form binds to and activates specific target proteins. The conversion of the inactive GDP-bound form to the active GTP-bound form is catalyzed by guanine nucleotide exchange factors (GEFs) and the reverse reaction is catalyzed by GTPase activating proteins (GAPs). The GTPase activating protein stimulates the GTP hydrolysis on the low-molecular-weight GTP-binding protein, cleaving GTP to phosphate and GDP.

A number of low-molecular-weight GTP-binding proteins have been already isolated, which have aroused a question as to their functional difference in the context of cells and tissues. To study this question, the activities of low-molecular-weight GTP-binding proteins have to be monitored in the living cells and tissues.

To know the activities of the low-molecular-weight GTP-binding proteins, the ratio of GTP-bound to GDP-bound forms of the low-molecular-weight GTP-binding proteins has to be determined. Currently, the following two methods are used routinely.

(1) ³²Pᵢ-labeling method: The low-molecular-weight GTP-binding proteins are purified from cells labeled with ³²Pᵢ. GTP and GDP bound to them are separated and quantified by thin layer chromatography (ref. 2).

(2) Pull-down method: Target-proteins that bind to the low-molecular-weight GTP-binding proteins are pre-bound to agarose beads and incubated with cell lysates. Since the GTP-bound form, but not GDP-bound form, binds to the target proteins with high affinity, only the GTP-bound form can be collected by this method. Then, the amount of GTP-bound forms is quantified by SDS-PAGE and immunoblotting (ref. 3).

However, both methods are applicable only to the cell lysates; therefore, no method have been applicable for the measurement of the activity of low-molecular-weight GTP-binding proteins in living cells.

It has been revealed that different biochemical reactions are processed not only at various intracellular organelles but also at various cytoplasmic localizations. Furthermore, the importance of low-molecular-weight GTP-binding proteins has been shown also in the higher brain function and the organ development. Thus, to monitor the activity of low-molecular-weight GTP-binding proteins in living cells and tissues are essential not only to understand the life, but also to develop a new drug. However, the biochemical methods described previously require cell lysates; therefore, it has been impossible to know where in the living cells or tissues the low-molecular-weight GTP-binding proteins are activated.

Meanwhile, green fluorescent protein (GFP) has been successfully used to visualize the localization of proteins in living cells (ref. 4). GFP is a group of proteins isolated from various animals such as Aequorea Victoria and emanates mostly green fluorescence and is extensively used to determine the intracellular localization of proteins. Groups of GFP include cyan-emitting mutant of GFP (CFP), yellow-emitting mutant of GFP (YFP), enhanced CFP (ECFP), enhanced YFP (EYFP), and enhanced blue-emitting mutant of GFP (EBFP), which are collectively called GFP hereafter. These GFPs are excited with lights of different wavelengths and emanated lights of longer wavelengths.

GFPs can be applicable to fluorescence resonance energy transfer (FRET) (ref. 5). FRET is a phenomenon as described below. Assuming two fluorescent proteins A and B, which emanate lights of emission wavelengths of λaem and λbem at excitation wavelengths of λaex and λbex, respectively. If molecule A is in close proximity of molecule B and if λaem overlaps λbex, excited energy of molecule A is transferred to molecule B, and the latter emanates a light of λbem. This phenomenon is called FRET and can be applicable to measure the distance between two fluorescent molecules. In this situation, molecule A and B are called as donor and acceptor, respectively.

Application of FRET includes detection of conformational change of proteins that are labeled with two fluorescent substances. Two sets of GFP-derived proteins, “EBFP and EGFP” and “ECFP and EYFP,” are known to provide such FRET pairs. For example, calcium concentration has been measured by a fusion protein consisting of EBFP, EGFP, and calmodulin. However, this single-molecule monitoring protein based on the technology of GFP and FRET is currently known only for the measurement of calcium and cAMP.

### SUMMARY OF THE INVENTION

The present invention aims at providing monitoring proteins which measure the activity of low-molecular-weight GTP-binding proteins in non-destructive manners, genes encoding said monitoring proteins, expression vectors containing said genes, cells and transgenic animals carrying said expression vectors, methods for measurement of the activity of low-molecular-weight GTP-binding proteins which use said monitoring proteins, particularly methods for the determination of the ratio of GTP-bound to GDP-bound low-molecular-weight GTP-binding proteins in living cells, and screening procedures for the regulatory substances of low-molecular-weight GTP-binding proteins.

In summary, the present invention relates to:

<1> Monitoring proteins for low-molecular-weight GTP-binding proteins consisting of: fused proteins, wherein the fused proteins include at least the low-molecular-weight GTP-binding protein, a target protein of said low-molecular-weight GTP-binding proteins, a GFP donor protein, and a GFP acceptor protein, whole or part of which are directly or indirectly connected each other, in a state wherein each of the protein retains its function,

<2> genes encoding said monitoring proteins for low-molecular-weight GTP-binding proteins,

<3> expression vectors which contain the genes described in<2>,

<4> cells transformed by the expression vectors described in<3>,

<5> transgenic animals which contain the expression vectors described in<3>,

<6> a method for measurement of the activity of the low-molecular-weight GTP-binding proteins comprising: the step of detecting FRET of the monitoring proteins for the low-molecular-weight GTP-binding proteins described in<1>,

<7> a method for measurement of the activity of the low-molecular-weight GTP-binding proteins comprising: the step of detecting FRET of the monitoring proteins for the low-molecular-weight GTP-binding proteins in the cells described in<4> or transgenic animals described in<5>,

<8> a screening method for the regulator of the activity of low-molecular-weight GTP-binding proteins comprising: (a) the step of culturing cells described in<4> in the presence of the specimens and (b) the step of measuring the activity change of low-molecular-weight GTP-binding proteins.

### BRIEF DESCRIPTION OF THE DRAWINGS

FIG. 1 shows an example of the regulation of the low-molecular-weight GTP-binding proteins. In the present figure, adducing Ras as an example of low-molecular-weight GTP-binding proteins, the regulation of low-molecular-weight GTP-binding proteins is schematically presented. The low-molecular-weight GTP-binding protein is inactive when it is bound to GDP. Guanine nucleotide exchange factor (GEF) promotes exchange of GDP with GTP, thereby activating the low-molecular-weight GTP-binding protein. The activated GTP-bound low-molecular-weight GTP-binding protein changes its conformation, thereby binding to and activating the target proteins. The activated low-molecular-weight GTP-binding protein hydrolyses GTP to GDP and Pᵢ in the presence of GTPase activating protein (GAP), thereby returning to the inactive GDP-bound state.

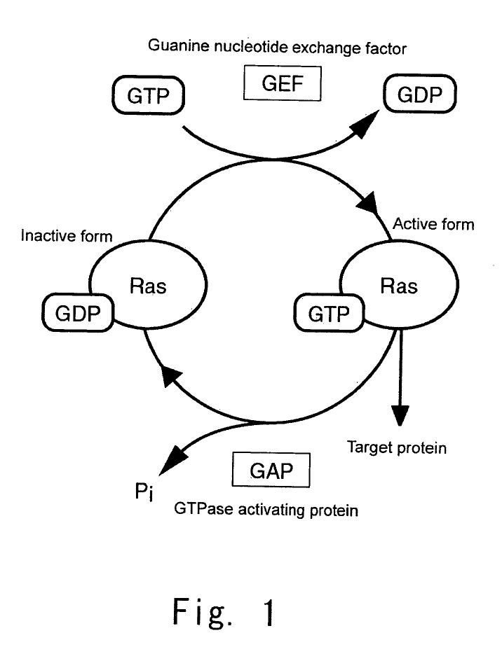

FIG. 2 shows an example of the principle of the measurement of the activity of low-molecular-weight GTP-binding protein based on FRET technology. In this figure, Ras and Raf are adduced as examples of low-molecular-weight GTP-binding proteins and their target proteins, respectively. Cyan-emitting mutant of GFP (CFP), which is adduced as an example of the GFP donor protein, emanates fluorescence of 475 nm by excitation at a wavelength of 433 nm. Meanwhile, yellow-emitting mutant of GFP (YFP), which is adduced as an example of the GFP acceptor protein, emanates fluorescence of 530 nm by excitation at a wavelength of 505 nm. In the present invention, CFP and YFP are used as the GFP donor and GFP acceptor proteins, respectively. As shown in the lower part of the FIG. 2, the energy of excited CFP is not effectively transferred to YFP before Ras activation, because YFP and CFP are positioned remotely. However, upon stimulation (for example, addition of epidermal growth factor (EGF)), activated Ras is induced to bind to the Ras-binding domain (RBD) of Raf, which brings YFP in close proximity of CFP, thereby causing the energy transfer from CFP to YFP, followed by the emission of 530-nm wavelength. Thus, by measuring the FRET efficiency before and after the stimulation (namely, before and after the Ras activation), the activity of Ras can be measured.

FIG. 3 shows an example of the structure of pRafras1722. pCAGGS, an expression vector used to express Rafras1722, has been reported previously. A cDNA encoding a fusion protein consisting of EYFP-Ras-RafRBD (Ras-binding domain)-ECFP from the amino-terminus is inserted downstream of CAG promoter as shown in the figure.

FIG. 4 shows an example of the nucleotide sequence and the amino-acid sequence decoded from the nucleotide sequence of the coding region of the plasmid pRafras1722.

FIG. 5 shows an example of the nucleotide sequence and the amino-acid sequence decoded from the nucleotide sequence of the coding region of the plasmid pRafras1722 (continued).

FIG. 6 shows an example of the nucleotide sequence and the amino-acid sequence decoded from the nucleotide sequence of the coding region of the plasmid pRafras1722 (continued).

FIG. 7 shows an example of the fluorescent profile of the expressed protein Rafras1722. HEK293T cells were transfected with pRafras1722 and an expression vector for guanine nucleotide exchange factor Sos (pCAGGS-mSos) or GTPase activating protein for Gaplm (pEF-Bos-Gap1m) by calcium phosphate coprecipitation method. Forty-eight hours after transfection, cells were lysed and cleared by centrifugation. Fluorescent intensity of the supernatant was examined with a fluorescent spectrometer from 450 to 550 nm wavelength range at an excitation wavelength of 433 nm. The right panel shows the fluorescent profiles of cells transfected with pRafras1722 and pCAGGS-mSos or pEF-Bos-Gap1m.

FIG. 8 shows an example of the correlation of the GTP/GDP ratio (GTP/ (GDP+GTP)) bound to the GTP-binding protein of the expressed Rafras1722 with the ratio of fluorescent intensity at 530 nm to fluorescent intensity at 475 nm (Em λ₅₃₀/Em λ475). HEK293T cells were transfected with pRafras1722 and various amounts of an expression vector for Sos (pCAGGS-mSos) or GTPase activating protein for Gap1m (pEF-Bos-Gaplm). Forty-eight hours after transfection, cells were labeled with ³²Pᵢ, and Rafras1722 was immunoprecipitated with anti-GFP antibody, followed by separation and quantitation of guanine nucleotides bound to Rafras1722 by thin layer chromatography. In parallel, cell lysates were analyzed for the fluorescent profiles to obtain the ratio of fluorescent intensity at 530 nm to 475 nm (Em λ₅₃₀/Em λ₄₇₅) at an excitation wavelength of 433 nm. Note that the ratio of fluorescent intensity increases with the increasing amount of GTP on Rafras1722.

FIG. 9 shows an example of the establishment of cell lines expressing Rafras1722. NIH3T3 cells were transfected with pRafras1722 to obtain a cell line, named 3T3-Rafras. Cells were lysed and analyzed by immunoblotting with anti-GFP antibody. Molecular-weight size-markers are shown at the left.

FIG. 10 shows an example of analysis of Ras activation using 3T3-Rafras cells. 3T3-Rafras cells were stimulated with EGF (1 μg/ ml) and fluorescent spectra (wavelength range from 450 nm to 550 nm) were obtained before and after stimulation.

FIG. 11 shows an example of the structure of pRai-chu311. The expression vector is as same as FIG. 3.

FIG. 12 shows an example of the nucleotide sequence and the amino-acid sequence decoded from the nucleotide sequence of the coding region of the plasmid pRai-chu311.

FIG. 13 shows an example of the nucleotide sequence and the amino-acid sequence decoded from the nucleotide sequence of the coding region of the plasmid pRai-chu311 (continued).

FIG. 14 shows an example of the nucleotide sequence and the amino-acid sequence decoded from the nucleotide sequence of the coding region of the plasmid pRai-chu311 (continued).

FIG. 15 shows an example of the fluorescent profile of expressed protein Rai-chu311. HEK293T cells were transfected with pRai-chu311 and an expression vector for guanine nucleotide exchange factor C3G (pCAGGS-C3G, described in ref. 9) or GTPase activating protein for rap1GAPII (pCAGGS-rap1GAPII, described in ref. 9) by calcium phosphate coprecipitation method. Forty-eight hours after transfection, cells were lysed and cleared by centrifugation. Fluorescent intensity of the supernatant was scanned with a fluorescent spectrometer from 450 to 550 nm wavelength range at an excitation wavelength of 433 nm. The right panel shows the fluorescent profile of cells transfected with pRai-chu311 and pCAGGS-C3G or pCAGGS-rap1GAPII.

FIG. 16 shows an example of the structure of pRai-chu158. The expression vector is same as FIG. 3.

FIG. 17 shows an example of the nucleotide sequence and the amino-acid sequence decoded from the nucleotide sequence of the coding region of the plasmid pRai-chu158.

FIG. 18 shows an example of the nucleotide sequence and the amino-acid sequence decoded from the nucleotide sequence of the coding region of the plasmid pRai-chu158 (continued).

FIG. 19 shows an example of the nucleotide sequence and the amino-acid sequence decoded from the nucleotide sequence of the coding region of the plasmid pRai-chu158 (continued).

FIG. 20 shows an example of the fluorescent profile of expressed protein Rai-chu158. HEK293T cells were transfected with pRai-chu158 and an expression vector for guanine nucleotide exchange factor CalDAG-GEFIII (pCAGGS-CalDAG-GEFIII, described in ref. 10) or GTPase activating protein for GAP1mI (pEF-Bos-GAPlm) by calcium phosphate coprecipitation method. Forty-eight hours after transfection, cells were lysed and cleared by centrifugation. Fluorescent intensity of the supernatant was scanned with a fluorescent spectrometer from 450 to 550 nm wavelength range at an excitation wavelength of 433 nm. The right panel shows the fluorescent profile of cells transfected with pRai-chu158 and pCAGGS-CalDAG-GEFIII or pEF-Bos-GAPlm.

FIG. 21 shows an example of the nucleotide sequence and the amino-acid sequence decoded from the nucleotide sequence of the coding region of the plasmid pRai-chu119.

FIG. 22 shows an example of the nucleotide sequence and the amino-acid sequence decoded from the nucleotide sequence of the coding region of the plasmid pRai-chu119 (continued).

FIG. 23 shows an example of the nucleotide sequence and the amino-acid sequence decoded from the nucleotide sequence of the coding region of the plasmid pRai-chu119 (continued).

FIG. 24 shows an example of the fluorescent profile of expressed protein Rai-chu119. HEK293T cells were transfected with pRai-chu119 or pRafras1722 and an expression vector for guanine nucleotide exchange factor Sos (pCAGGS-mSos) by calcium phosphate coprecipitation method. Twenty-four hours after transfection, temperature of the cell culture was changed to 33° C. or 40° C. After further 24 hrs incubation, cells were lysed and cleared by centrifugation. Fluorescent intensity of the supernatant was scanned with a fluorescent spectrometer from 450 to 550 nm wavelength range at an excitation wavelength of 433 nm. The right panel shows the fluorescent profile of cells transfected with pRafras1722 or pRai-chu119 and pCAGGS-mSos. Rai-chu119 responded more efficiently to the guanine nucleotide exchange factor than did the wild-type (Rafras1722).

FIG. 25 shows an example of the time course of fluorescent intensities of ECFP and EYFP after the addition of epidermal growth factor (EGF). Cells were illuminated at a wavelength of 430 nm to obtain time-lapse fluorescence images at a wavelength of 475 nm and 530 nm, which were then used to determine the fluorescent intensities of ECFP and EYFP, respectively.

FIG. 26 shows an example of the change in the fluorescent intensities of ECFP and EYFP of Rafras1722 by the expression of various kinds of guanine nucleotide exchange factors and GTPase activating proteins. HEK293T cells were transfected with pRafras1722 and expression vectors for guanine nucleotide exchange factors or GTPase activating proteins by the calcium coprecipitation method. Twenty-four hours later, cells were lysed and cleared by centrifugation. By using the supernatant, fluorescent intensities at 475 nm and 530 nm were determined at an excitation wavelength of 433 nm with a fluorescent spectrometer. The ratio of the latter to the former (fluorescence ratio) is shown in the graph.

FIG. 27 shows an example of the change in the fluorescent intensities of ECFP and EYFP of Rai-chu404 by the expression of various kinds of guanine nucleotide exchange factors and GTPase activating proteins. HEK293T cells were transfected with pRai-chu404 and expression vectors for guanine nucleotide exchange factors or GTPase activating proteins by calcium coprecipitation method. Twenty-four hours later, cells were lysed and cleared by centrifugation. By using the supernatant, fluorescent intensities at 475 nm and 530 nm were determined at an excitation wavelength of 433 nm with a fluorescent spectrometer. The ratio of the latter to the former (fluorescence ratio) is shown in the graph.

FIG. 28 shows an example of the time course and intracellular distribution of the fluorescence ratio of EYFP to ECFP in COS1 cells transfected with pRai-chu101X or pRai-chu404X and stimulated with EGF. COS1 cells transfected with pRai-chu101X or pRai-chu404X were cultured for 24 hrs. The medium was changed to MEM without phenol-red and serum before imaging. Cell images were obtained with an imaging system consisting of Metamorph image analyzing software (Roper Scientific Japan) and inverted fluorescent microscope Axiovert 100 (Carl Zeiss) equipped with Xenon lamp, revolving filter changers for excitation filters and emission filters (LUDL electronic), and high sensitivity cooled CCD camera Micromax 450 (Photometrix). Cells were illuminated with an excitation wavelength of 430 nm and fluorescent images of ECFP donor protein at 475 nm and EYFP acceptor protein at 530 nm were obtained every 30 sec. After data acquisition, from blue to red colors was assigned to each pixel of the digital images, depending on the levels of EYFP/ECFP fluorescence ratios. Meanwhile, the intensity of ECFP is assigned to the intensity of each pixel. From time-lapse images, only the images at the indicated time point are shown. By the simulation of EGF, the fluorescence ratio, which reflects the FRET efficiency, gradually increases from the periphery to the center of the cells expressing Rai-chu101X. In contrast, the activity of Rap1 increases from the center to the periphery of the cells expressing Rai-chu404X. Thus, the invented monitoring proteins can monitor the spatio-temporal change in the activity of Ras-family G proteins.

FIG. 29 shows an example of the time course and intracellular distribution of the fluorescence ratio of EYFP to ECFP in subconfluent COS1 cells transfected with pRai-chu101X and stimulated with EGF. Experiments were performed similarly to FIG. 28 except that subconfluent COS1 cells were used. Upon stimulation with EGF, the fluorescence ratio, which reflects the FRET efficiency, increases from the periphery where cells are not in contact with the neighboring cells. In contrast, at the region where the COS cells are in contact with the neighboring cells, the increase in FRET efficiency is suppressed.

FIG. 30 shows an example of the time course and intracellular distribution of the fluorescence ratio of EYFP to ECFP in PC12 cells transfected with pRai-chu101X or pRai-chu404X and stimulated with nerve growth factor. PC12 cells transfected with pRai-chu101X or pRai-chu404X were cultured more than 24 hrs. After changing the medium to MEM without serum and phenol-red, cells were stimulated with nerve growth factor and observed as in FIG. 29. Only the figures at the indicated time points are shown. Upon stimulation of Rai-chu101X expressing cells with nerve growth factor, the fluorescence ratio, which reflects the FRET efficiency, increases from the periphery to the center. Then, after 180 min, when the neuronal extension is visible, the increase in FRET efficiency is limited mostly at these extended neurites. In contrast, in the cells expressing Rai-chu404X, the activity increases from the center to the periphery and is suppressed at the differentiated extended neurites. This observation indicates that Ras is activated from the periphery and Rap1 from the center during the neuronal differentiation, and that high Ras activity is maintained at the extended neurites. This observation further indicates that each Ras-family G protein is activated at different intracellular localization and demonstrates the usefulness of the invented monitoring proteins to obtain the spatio-temporal information of the activity of Ras-family G proteins.

FIG. 31 shows an example of the structure of pRai-chu1011X. The basal vector is as same as FIG. 3.

FIG. 32 shows an example of the structure of pRai-chu1054X. The basal vector is as same as FIG. 3.

FIG. 33 shows an example of the structure of pRai-chu1212X. The basal vector is as same as FIG. 3.

FIG. 34 shows an example of the fluorescence profile of Rai-chu1011X (wild type), Rai-chu1012X (activated form), and Rai-chu1013X (inactive form). HEK293T cells were transfected with pRai-chu1011X, pRai-chu1012X, or pRai-chu1013X by the calcium phosphate method. Forty-eight hours later, cells were lysed and centrifuged to obtain supernatant, which was analyzed with a fluorescent spectrometer to obtain the fluorescent profiles from 450 nm to 550 nm.

FIG. 35 shows an example of the fluorescence profile of Rai-chu1054X (wild type) and Rai-chu1052X (activated form). HEK293T cells were transfected with pRai-chu1054X or pRai-chu1052X by calcium phosphate method. Forty-eight hours later, cells were lysed and centrifuged to obtain supernatant, which was then analyzed by spectrometer to obtain the fluorescent profiles from 450 nm to 550 nm.

FIG. 36 shows an example of the fluorescence profile of Rai-chu1212X (wild type) and Rai-chu1220X (activated form). HEK293T cells were transfected with pRai-chu1212X or pRai-chu1220X by calcium phosphate method. Forty-eight hours later, cells were lysed and centrifuged to obtain supernatant, which was then analyzed with a fluorescent spectrometer to obtain the fluorescent profiles from 450 nm to 550 nm.

FIG. 37 shows an example of the time course and intracellular distribution of the fluorescence ratio of EYFP to ECFP in COS1 cells transfected with pRai-chu1011X and stimulated with EGF. Experiments were performed similarly to FIG. 28. Upon stimulation with EGF, the fluorescence ratio, which reflects the FRET efficiency, increases diffusely within one minute, followed by increase at the membrane ruffles and decrease in the central region. This spatio-temporal distribution of Rac activity is remarkably different from those of Ras and Rap1 examined by Ra-chu101X or Rai-chu404X, respectively. This observation indicates that the invented monitoring proteins can obtain the spatio-temporal information of the activities of Rho-family G proteins.

### DESCRIPTION OF THE PREFERRED EMBODIMENTS

The invented monitoring proteins for the activity of low-molecular-weight GTP-binding proteins, called monitoring proteins hereafter, utilize the GTP-dependent binding to the target proteins by the low-molecular-weight GTP-binding proteins and provide extremely useful tools which can measure the activity low-molecular-weight GTP-binding proteins in living cells. The invented monitoring proteins consist of low-molecular-weight GTP-binding protein, its target protein, GFP donor protein, and GFP acceptor protein, which are ligated directly or indirectly so that each component functions properly. Therefore, these fusion proteins have structures that amino acid sequences of said proteins are ligated directly or indirectly. Notably, each component does not need to consist of the full-length protein if it retains its function.

In this disclosure, where it should be exactly expressed as “a part of protein,” it is simply called as “a protein”; Adducing target protein as an example, when it should be called as “a part of target protein,” it is simply expressed as “a target protein.”

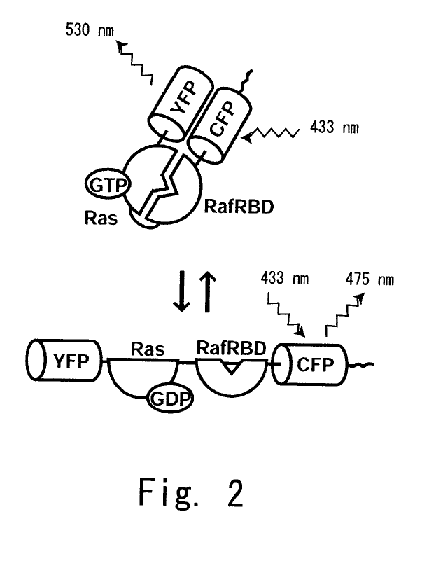

In the monitoring protein described in this disclosure, the binding of GTP to the low-molecular-weight GTP-binding protein, or exchange of GDP with GTP, induces intramolecular binding of the low-molecular-weight GTP-binding protein to the target protein, changing the level of FRET efficiency. FIG. 2 shows the schematic representation of the principle of the measurement of the activity of low-molecular-weight GTP-binding proteins with the invented monitoring proteins. In this invention, the FRET efficiency means the ratio of the intensity of acceptor fluorophore to the intensity of the acceptor fluorophore. This will be described in detail in the following paragraphs.

For the FRET measurement, the following three factors demand consideration. I) Overlap of the emission wavelength of the GFP donor and the excitation wavelength of the GFP acceptor. II) Distance between the donor and acceptor. III) Moment of the emission from donor and the moment of the excitation of the acceptor. Furthermore, by the structural tension caused by the fused proteins, GFPs may not form chromophore efficiently. Therefore, a probe wherein the energy is transferred efficiently from the GFP donor to the GFP acceptor by FRET can be constructed only after fulfilling many strict conditions. However, the conditions wherein FRET is always observed has not been reported and usually, construction of such probes require enormous efforts. In other words, a FRET-based probe cannot be easily constructed on the known techniques and can be prepared only after many try-and-error experiments and many sophisticated experiments. The invented monitoring proteins are generated after such efforts to obtain the desired effects. In this probe the specific binding of GTP-bound low-molecular-weight GTP-binding protein to the target protein is designed to greatly change the level of FRET efficiency; therefore it is extremely useful in many applications.

The order of the components of the probes can be selected by the change in the level of FRET efficiency after the activation of the low-molecular-weight GTP-binding proteins, which will be simply called the change in FRET efficiency. Larger the change in FRET efficiency, more sensitive is the detection of the activation of the low-molecular-weight GTP-binding proteins. The most desirable aspects of the probes include that the carboxyl-terminus of the low-molecular-weight GTP-binding proteins is bound directly or indirectly to the amino-terminus of the target protein (1) and that the carboxyl-terminus of the target protein is bound directly or indirectly to the amino-terminus of low-molecular-weight GTP-binding proteins (2). Particularly, when the low-molecular-weight GTP-binding proteins belong to the Ras-family, the aspect (1) is preferable; when the low-molecular-weight GTP-binding proteins belong to the Rho-family, the aspect (2) is preferable. GFP acceptor and GFP donor proteins are ligated directly or indirectly to either the amino-terminus or the carboxyl-terminus of the G protein-target protein complex. In particularly preferable aspects, the G protein-target protein complex binds to GFP acceptor at its amino-terminus and to GFP donor at its carboxyl-terminus. Therefore, the invented monitoring protein preferably consists of, from the amino-terminus, GFP acceptor protein, low-molecular-weight GTP-binding protein, its target protein, and GFP donor protein, which are bound to each other directly or indirectly. When it is called indirectly, it means that the proteins are linked each other with a peptide spacer as will be described later.

There is no restriction in the kind of ωthe low-molecular-weight GTP-binding proteins in the invented monitoring protein; however, from the view of its usefulness, preferably it should belong to Ras-superfamily G proteins, particularly to Ras-family or Rho-family. More preferably, low-molecular-weight GTP-binding protein should be chosen among H-Ras, K-Ras, N-Ras, R-Ras, Rap1A, Rap1B, Rap2A, and Rap2B that belong to the Ras-family, or RhoA, RhoB, RhoC, Rac1, Rac2, and Cdc42 that belong to the Rho-family.

There is no restriction in the species of target proteins, if they bind to the low-molecular-weight GTP-binding proteins in a GTP-dependent manner. For the viewpoint of usefulness, they are Raf and RalGDS for Ras-family and Pak or mDia for the Rho-family.

Furthermore, as pairs of low-molecular-weight GTP-binding protein and its target protein, from the viewpoint of usefulness and specificity, the following are preferable: the low-molecular-weight GTP-binding protein is H-Ras and the target protein is Raf, the low-molecular-weight GTP-binding protein is Rap1A and the target protein is RalGDS, the low-molecular-weight GTP-binding protein is Rac1 and the target protein is Pak, the low-molecular-weight GTP-binding protein is Cdc42 and the target protein is Pak, and the low-molecular-weight GTP-binding protein is RhoA and the target protein is mDia.

Any of the GFP-related proteins can be used as the GFP acceptor protein. From the functional viewpoint, EGFP and EYFP are preferable. Similarly, any of the GFP-related proteins can be used as GFP donor protein and from the functional viewpoint, ECFP and EBFP are preferable.

From the viewpoints of usefulness, specificity, and sensitivity, most preferable combinations of the constituents of the probes are as following: (1) The low-molecular-weight GTP-binding protein is H-Ras, the target protein is Raf, GFP donor protein is ECFP, and GFP acceptor protein is EYFP. (2) The low-molecular-weight GTP-binding protein is Rap1A, the target protein is RalGDS, GFP donor protein is ECFP, and GFP acceptor protein is EYFP. (3) The low-molecular-weight GTP-binding protein is Rac1, the target protein is Pak, GFP donor protein is ECFP, and GFP acceptor protein is EYFP. (4) The low-molecular-weight GTP-binding protein is Cdc42, the target protein is Pak, GFP donor protein is ECFP, and GFP acceptor protein is EYFP. (5) The low-molecular-weight GTP-binding protein is RhoA, the target protein is mDia, GFP donor protein is ECFP, and GFP acceptor protein is EYFP.

From the viewpoint of the change in FRET efficiency, the preferable orders of the low-molecular-weight GTP-binding protein, target protein, GFP donor protein, and GFP acceptor protein in the invented probes are as following: EYFP-H-Ras-Raf-ECFP, EYFP-Rap1A-RalGDS-ECFP, EYFP-Pak-Rac1-ECFP, EYFP-Pak-Cdc42-ECFP, and EYFP-mDia-RhoA-ECFP. Notably, the orders of EYFP and ECFP can be changeable.

The low-molecular-weight GTP-binding protein does not necessarily consist of full-length peptide and can be a part of low-molecular-weight GTP-binding protein, if it can bind to the target protein. This property of a part of low-molecular-weight GTP-binding protein can be tested, for example, by examining its binding to target proteins after in vitro loading of GTP by any known methods. The binding of a part of low-molecular-weight GTP-binding protein to the target protein can be detected, for example, by immunoprecipitating the target protein and detecting the part of low-molecular-weight GTP-binding protein by immunoblotting. Followings are the examples of the parts of low-molecular-weight GTP-binding protein: amino-acids 1 to 180, or preferably 1 to 172, of H-Ras and Rap1; amino-acids 1 to 204, or preferably 28 to 204, of R-Ras; amino-acids 1 to 177 of Rac1; amino-acids 1 to 176 of Cdc42 and RhoA.

Sometimes, the change in FRET efficiency can be increased by trimming the amino- and/or carboxyl-terminal regions of the low-molecular-weight GTP-binding protein. Therefore, “a part of low-molecular-weight GTP-binding protein” includes those with at least one amino-acid deletion, preferably 1 to 28, more preferably 17 to 28 amino-acid deletions. For example, the change in FRET efficiency is larger in the probe with the carboxyl-terminal deletion to amino acid 170 than one to amino acid 180. Therefore, the carboxyl-terminus of low-molecular-weight GTP-binding protein should be trimmed for, at least one, preferably 9 to 20, more preferably 17 amino acids.

The amino-terminal and carboxyl-terminal regions generally indicate up to 30 amino-acid regions from either amino-terminus or carboxyl-terminus of the low-molecular-weight GTP-binding protein.

Similarly, the target protein does not necessarily consist or full-length peptide and can be a part of the target protein, if it can bind to low-molecular-weight GTP-binding protein. Notably, the nature of a part of target protein can be examined similarly as described for low-molecular-weight GTP-binding protein. Followings are such examples: in case of Raf (Genbank/EMBL accession number: X03484), preferably the Ras-binding region (amino acid 51 to 204), more preferably 51 to 131; in case of RalGDS (Genbank/EMBL accession number: U14417), preferably amino acid 202 to 309, more preferably amino acid 211 to 297; in case of Pak1 (Genbank/EMBL accession number: NM002576), amino acid 68 to 150; in case of mDia1 (Genbank/EMBL accession number: E17361), amino acid 68 to 240, more preferably 68 to 180.

Meanwhile, GFP donor and/or GFP acceptor protein does not necessarily constitute of full-length peptide and can be a part of the target protein, only if it can be used as FRET pairs. Sometimes, trimming the carboxyl-terminal regions of these proteins increases the change in FRET efficiency. For example, GFP donor and/or GFP acceptor protein preferably possesses at least one, more preferably one to eleven amino acids deletion. In case of EYFP, its carboxyl region may have preferably at least one, more preferably one to eleven amino-acids deletion. In case of ECFP, its carboxyl region may have preferably at least one, more preferably one to eleven amino acids deletion. Here the carboxyl-terminal region is defined as the amino acid region of 1 to 20, preferably up to 11 amino acid from the carboxyl terminus of GFP-related proteins. Whether the trimmed GFP proteins function as FRET pairs can be examined as follows: The GFP proteins are expressed in E. coli and then the fluorescent spectrum of the cell lysates are obtained by using the cell lysates.

Furthermore, the GFP donor and/or acceptor proteins can possess mutations. These mutations can be introduced to any amino acid regions as far as they do not inhibit FRET. One aspect of such mutation is a GFP mutant (Phe64Leu, Val68Leu, Ser72Ala, Ile67Thr). Introduction of these mutations are preferable because they may increase the efficiency of fluorophore maturation or change in FRET efficiency.

The mutation can also be introduced into the low-molecular-weight GTP-binding protein or target proteins. For example, by introducing a point mutation, the sensitivity to guanine nucleotide exchange factors or GTPase activating proteins can be increased. These mutations can be introduced to at any amino acid regions as far as they do not inhibit the intramolecular binding of low-molecular-weight GTP-binding protein and target proteins. Aspects of such mutations include amino-acid substitution, insertion, and/or deletion. For example, Ile36Leu mutation in H-Ras amino-acid sequence increases the sensitivity of the probe to the GTPase activating protein. As a result, the dynamic range of the probe can be increased. These mutant H-Ras proteins are preferably used in the invented monitoring proteins. These mutations can be easily introduced by using either restriction enzymes or by PCR.

In the invented monitoring protein, the spatial arrangement of each component protein affects its function. By changing the spatial arrangement, the change in FRET efficiency can be remarkably increased. For example, by the insertion of a spacer peptide between the protein components, the change in FRET efficiency can be modulated. To increase the change in FRET efficiency, such spacers are preferably inserted between the low-molecular-weight GTP-binding protein and the target protein. The length of spacer peptide, which can consist of any amino acids, is preferably between 1 to 30, more preferably 1 to 10. By inserting these peptides, the change in FRET efficiency and/or the folding of GFP can be enhanced. For the proper conformational arrangement, the spacer peptides may preferably consist of many glycine residues.

Another preferable aspect of the monitoring proteins is that the monitoring protein is fused to other peptides or proteins at either the amino-terminus or the carboxyl-terminus. Particularly, by fusing intracellular localization signals such as endoplasmic reticulum-localization signal or membrane localization signal, the monitoring proteins can measure the local activity of low-molecular-weight GTP-binding proteins. Furthermore, as will be described later, the monitor can measure the local ratio of GTP-bound to GDP-bound low-molecular-weight GTP-binding proteins.

In the invented monitoring proteins, the activation of low-molecular-weight GTP-binding protein by GTP loading will cause the intramolecular binding of the low-molecular-weight GTP-binding protein to the target protein, thereby inducing the conformational change of the probe, thereby changing the relative direction and distance between the GFP donor and the GFP acceptor proteins. Therefore, by the excitation at predetermined wavelength, the increase in FRET efficiency from the donor to acceptor proteins can be monitored. Such change in FRET efficiency is affected by the positioning of GFP donor and GFP acceptor proteins after the conformational change of the probe. For example, shortening of the distance between GFP donor and GFP acceptor proteins will increase in FRET efficiency, and the lengthening of it will decrease the FRET efficiency. Dynamic range of the FRET efficiency, in the other words, the difference between the maximum and the minimum FRET efficiency, can be tuned to the desired level by inserting spacer peptides, depending on the property of constituent proteins.

This invention also provides the genes encoding the invented monitoring proteins. Such genes can be constructed by obtaining the sequence information from Genbank etc., by PCR amplification, or by using restriction enzymes and ligase.

Followings are names and Genbank/EMBL accession numbers of proteins used preferably as constituents of monitoring proteins. Accession numbers are shown in the parenthesis.

(1) Low-Molecular-Weight GTP-Binding Proteins

H-Ras (V00574), K-Ras (L00045- L00049), N-Ras (L00040-L00043), R-Ras (M14948, M14949), Rap1A (X12533), Rap1B (X08004), Rap2A (X12534),Rap2B (X52987), RhoA (L25080), RhoB (X06820), RhoC (X06821), Rac1(M29870), Rac2 (NM002872), Rac3 (NM005052), Cdc42 (M57298)

(2) Target Protein

Raf (X03484), RalGDS (U14417), Pak1 (NM002576), mDia1 (E17361)

(3) GFP Donor and Acceptor Proteins

EGFP (U76561), EYFP (U73901), ECFP (AB041904)

EBFP described in ref. 6 carries the following three mutations:

Phe64Leu, Tyr66His, Tyr145Phe.

The present invention also provides the expression vectors encoding the genes. Such vectors are obtained by inserting the genes of the monitoring proteins into any known prokaryotic expression vectors including pGEX-2T (Amersham), eukaryotic expression vectors including pCAGGS (ref. 7), or viral vectors including pShuttle (Clontech). As an expression vector, expression plasmids are used preferably.

The present invention also provides cells or transgenic animals carrying the expression vectors. Such cells can be obtained by introducing the expression vectors into the cells. There is no restriction in the method of introducing the genes into the cells, including calcium phosphate coprecipitation method, lipofection, or electroporation. Any prokaryotic or eukaryotic cells can be used as the host. Followings are some examples of eukaryotic cells; human embryonic kidney cell HEK293T, monkey kidney cell COS, human umbilical venous endothelial cell; and prokaryotic cells, including E. coli. Meanwhile, by microinjecting the expression vector into mouse fertilized eggs, transgenic mouse can be obtained.

The present invention also provides the method for the measurement of the activity of low-molecular-weight GTP-binding proteins. In this method, by measuring the FRET efficiency of said monitoring protein, the activity of low-molecular-weight GTP-binding proteins can be measured. Moreover, by measuring the FRET of said transformed cells or transgenic animals, the activity of low-molecular-weight GTP-binding proteins in these cells and animals can be measured. By preparing a calibration curve of GTP/GDP ratio of the low-molecular-weight GTP-binding proteins against FRET efficiency of the probe, the data on the FRET efficiency can be correlated with the data of GTP/GDP on the low-molecular-weight GTP-binding proteins.

Followings are such examples.

(1) A Method Using Spectrometer

Cells that can express monitoring proteins are cultured in the condition wherein the monitoring proteins are expressed. Cells can be lysed by any methods, preferably by using buffer containing Triton X-100. The cell lysates are illuminated at an excitation wavelength of GFP donor protein (ex. 433 nm) and spectrogram is obtained with any known spectrometers. Based on the spectrogram, for example, the ratio of fluorescent intensity of donor protein at a wavelength of 475 nm vs fluorescent intensity of acceptor protein at a wavelength of 530 nm ((fluorescent intensity at an wavelength of 530 nm)/ (fluorescent intensity at an wavelength of 475 nm)) is calculated to estimate the FRET efficiency. Because the FRET efficiency after GTP loading to the low-molecular-weight GTP-binding proteins (namely, activation of low-molecular-weight GTP-binding proteins) is higher than that before GTP binding, the FRET efficiency can be used to measure the activation of low-molecular-weight GTP-binding proteins. The activation of low-molecular-weight GTP-binding proteins can be induced by co-expressing guanine nucleotide exchange factor expression vector such as pCAGGS-Sos (ref. 9) or by stimulating the cells with growth factors such as EGF. Similarly, inactivation of low-molecular-weight GTP-binding proteins can be induced by co-expressing expression vectors for GTPase activating proteins such as pEF-Bos-GAPlm (ref. 9). Meanwhile, since the FRET efficiency is influenced by the distance and direction of the GFP donor and the GFP acceptor, the change in protein conformation can also be detected by the change in FRET efficiency.

(2) A Method Using Fluorescence Microscope

The change in FRET efficiency before and after the activation of low-molecular-weight GTP-binding proteins can be directly examined by observing the invented cells or transgenic animals expressing monitoring proteins with any fluorescence microscope. The activation and inactivation of low-molecular-weight GTP-binding proteins can be induced similarly to (1).

Any microscope can be used; however, inverted fluorescence microscope (Carl Zeiss, Axiovert 100) equipped with revolving filter changers containing excitation and emission filters and high sensitivity cooled CCD camera. More preferably, the filter changers and CCD camera are controlled by Metamorph imaging software (Roper Scientific Japan).

The cells or animals are illuminated at the excitation wavelength of GFP donor protein and the image is obtained at the emission wavelength of the donor protein. Then, the image is obtained at the wavelength of the fluorescence of the acceptor protein. By calculating the ratio of the intensities of the both images, FRET efficiency at each pixel can be obtained. The calibration of FRET data with GTP/GDP ratio can be performed as following. First, various activation levels of low-molecular-weight GTP-binding protein is achieved by expressing various amounts of guanine nucleotide exchange factor such as Sos. Then, FRET efficiency in these cells is examined with a fluorescent microscope by the method. In parallel, similarly-prepared cells are lysed and used to measure the ratio of GTP-bound to GDP-bound low-molecular-weight GTP-binding proteins as described (ref. 2). Lastly, the data of FRET efficiency are plotted against the GTP/GDP ratio of the monitoring proteins. In other words, both FRET efficiency and GTP/GDP ratio of the monitoring proteins are measured in various conditions, which data are used to prepare calibration curve. By this method, by simply observing the cells or animals with a fluorescent microscope and measuring the FRET efficiency, the GTP/GDP level at each time point and each place can be determined. Therefore, this method allows us to know the activation status of low-molecular-weight GTP-binding protein very easily in living cells, and furthermore, these data can be correlated with the GTP/GDP ratio. Similar method for preparing the calibration curve can be also applicable to the method (1).

The present invention provides the monitoring proteins that envision non-destructive measurement of the activation status of low-molecular-weight GTP-binding proteins, and also its genes etc. The present invention also provides cells and transgenic animals that express and encode the useful monitoring protein, and also the method to measure the activity of low-molecular-weight GTP-binding proteins. Therefore, this invention enables us to know the activation status of low-molecular-weight GTP-binding proteins by non-destructive methods. These features will have a great benefit not only in the field of bioscience but also in the development of drugs, for example, therapeutic and prophylactic drugs for cancer, autoimmune disease, and allergic disease.

Another aspect of this invention is the screening methods for the substances which regulate the activity of low-molecular-weight GTP-binding proteins. Namely,

(a) procedure wherein substances are incubated with cells carrying the expression vector for and expressing the monitoring protein of low-molecular-weight GTP-binding protein, and

(b) procedure wherein the activity change of low-molecular-weight GTP-binding protein is detected.

According to this screening method, substances or their salts that can change the activity of low-molecular-weight GTP-binding protein (namely, the regulatory substances of low-molecular-weight GTP-binding protein) can be effectively screened by preparing cells that express the monitoring proteins for the low-molecular-weight GTP-binding protein and constructing bioassay system. The subject can be any materials, but is preferably peptide, protein, non-peptide materials, synthetic materials, and fermented materials.

The invented screening procedure can be performed (i) in the presence of the activators of low-molecular-weight GTP-binding proteins, or (ii) in the absence of the activator of low-molecular-weight GTP-binding proteins. Here, the activator of low-molecular-weight GTP-binding proteins means substances that active low-molecular-weight GTP-binding proteins, for example, cell growth factors such as epidermal growth factor, or cytokines such as interleukin; however, the activator is not limited to these materials. The regulatory substances of low-molecular-weight GTP-binding proteins can be detected as materials that either increase or decrease the activity of low-molecular-weight GTP-binding proteins in case of method (i), and as materials that increase the activity of low-molecular-weight GTP-binding proteins in case of method (ii).

Detailed description of the invented screening procedure is as follows. In the presence or absence of the activator, the cells expressing the invented monitoring proteins are incubated with the substances in case of procedure (a) (aspect 1). There is no limitation in the method of incubation, for example, the cells can be cultured in the presence of the substances. In parallel, as a control, the cells are kept in the same condition without incubating with the substances (aspect 2). Then, in procedure (b), the activity of low-molecular-weight GTP-binding protein is measured. By comparing the activity measured in aspect 1 with that in aspect 2, the regulatory substances of low-molecular-weight GTP-binding protein can be screened. The activity of low-molecular-weight GTP-binding protein is measured by quantitating the FRET efficiency.

In conclusion, the substances that enhance the activity of low-molecular-weight GTP-binding proteins in case (i) is the regulatory substances that increase the activity of low-molecular-weight GTP-binding proteins, and, in contrast, the materials that suppress the activity of low-molecular-weight GTP-binding proteins in case (i) is the regulatory substances that decrease the activity of low-molecular-weight GTP-binding proteins. Furthermore, the substances that enhance the activity of low-molecular-weight GTP-binding proteins in case (ii) are the regulatory substances that increase the activity of low-molecular-weight GTP-binding proteins.

### REFERENCES

Followings are the list of references described in this disclosure.

1. Bos, J. L. 1997. Ras-like GTPases. Biochim. Biophys. Acta 1333:M19-M31.

2. Satoh, T. and Y. Kaziro. 1995. Measurement of Ras-bound guanine nucleotide in stimulated hematopoietic cells. Method. Enzymol. 255:149-155.

3. Franke, B., J. W. N. Akkerman, and J. L. Bos. 1997. Rapid Ca2+-mediated activation of Rap1 in human platelets. EMBO J. 15:252-259.

4. Tsien, R. Y. and A. Miyawaki. 1998. Seeing the machinery of live cells. Science 280:1954-1955.

5. Pollok, B. A. and R. Heim. 1999. Using GFP in FRET-based applications. Trends Cell Biol. 9:57-60.

6. Miyawaki, A., J. Llopis, R. Heim, J. M. McCaffery, J. A. Adams, M. Ikura, and R. Y. Tsien. 1997. Fluorescent indicators for Ca2+ based on green fluorescent proteins and calmodulin. Nature 388:882-887.

7. Niwa, H., K. Yamamura, and J. Miyazaki. 1991. Efficient selection for high-expression transfectants with a novel eukaryotic vector. Gene 108:193-200.

8. DeClue, J. E., J. C. Stone, R. A. Blanchard, A. G. Papageorge, P. Martin, K. Zhang, and D. R. Lowy. A ras effector domain mutant which is temperature sensitive for cellular transformation: interactions with GTPase-activating protein and NF-1. Mol.Cell Biol. 11:3132-3138, 1991.

9. Ohba, Y., N. Mochizuki, S. Yamashita, A. M. Chan, J. W. Schrader, S. Hattori, K. Nagashima, and M. Matsuda. Regulatory proteins of R-Ras, TC-21/R-Ras2, and M-Ras/R-Ras3. J. Biol. Chem. 275:20020-20026, 2000.

10.Yamashita, S., N. Mochizuki, Y. Ohba, M. Tobiume, Y. Okada, H. Sawa, K. Nagashima, and M. Matsuda. GalDAG-GEFIII activation of Ras, R-Ras, and Rap1. J. Biol. Chem. 275:25488-25493, 2000.

11. T. Gotoh, S. Hattori, S. Nakamura, H. Kitayama, M. Noda, Y. Takai, K. Kaibuchi, H. Matsui, O. Hatase, H. Takahashi, T. Kurata, and M. Matsuda. Identification of Rap1 as a target for Crk SH3 domain-binding guanine nucleotide-releasing factor, C3G. Mol.Cell.Biol. 15:6746-6753, 1995.

### EXAMPLES

Hereinafter, this invention will be described with examples; however, the content of this invention shall not be limited to these examples. Note that human H-Ras, human c-Raf1, human Rap1A, human RalGDS, human R-Ras, human Rac1, human Cdc42, human RhoA, human Pak1, and human mDia1 will be called simply as Ras, Raf, Rap1A, RalGDS, R-Ras, Rac1, Cdc42, RhoA, Pak1, and mDia1, respectively.

### Eample 1

### Measurement of Ras Activity by the Use of Rafras1722

(1) Construction of a Gene Encoding a Chimera of Ras and Raf

(i) Amplification of Ras Gene

Using Ras cDNA (Genbank/EMBL accession number: V00574) as a template, sense primer hRasXh (5′-CTCGAGATGACGGAATATAAGCTGGTGGTG-3″) (sequence number: 1), anti-sense primer Rasl72Raf (5′-AGTGTTGCTTGTCTTAGAAGGGGTACCACCTCCGGAGCCGTTC AGCTTCCGCAGCTTGTG-3′) (sequence number: 2), and heat stable DNA polymerase Pfx (Gibco-BRL, Bethesda, U.S.A.), DNA fragment corresponding to amino acid 1 to 172 of Ras was amplified by polymerase chain reaction (PCR).

Sense primer hRasXh consists of the underlined recognition sequence of restriction enzyme XhoI and a DNA sequence corresponding to the amino acid 1 to 8 of Ras. Meanwhile, anti-sense primer Ras172Raf consists of a complementary DNA sequence of Raf corresponding to amino-terminal region of the Ras binding domain (amino acid 61 to 67), spacer sequence (underlined), and DNA sequence of Ras corresponding to amino acid 166 to 172.

(ii) Amplification of Raf Gene

Using Raf cDNA (Genbank/EMBL accession number: X03484) as a template, sense primer RafRBD-F1 (5′-GGTACCCCTTCTAAGACAAGCAACACT -3″)(sequence number: 3), anti-sense primer RafRBDn2 (5′-GCGGCCGCCCAGGAAATCTACTTGAAGTTC -3′) (sequence number: 4), and said Pfx, DNA corresponding to amino acid 51 to 131 of Raf were amplified by PCR.

Sense primer RafRBD-F1 consists of the underlined recognition sequence of restriction enzyme KpnI and a DNA sequence corresponding to the amino acid 51 to 57 of Raf. Anti-sense primer RafRBDn2 consists of a complementary DNA sequence of Raf corresponding to the carboxyl-terminal region of the Ras binding domain (amino acid 125 to 131).

Using a mixture of the amplified DNA fragments described in (i) and (ii) as templates, sense primer hRasXh, anti-sense primer RafRBDn2, and said Pfx, DNA of a chimera of Ras and Raf was amplified by PCR. Then, the obtained DNA fragment was cloned into pCR-bluntII-TOPO (Invitrogen), followed by transformation of E. coli. Then, the E. coli was cultured and plasmids were prepared by the SDS-alkaline method.

(2) Construction of pFret2, an Expression Vector Encoding EYFP and ECFP

(i) Construction of pCAGGS-P7

The multiple cloning site of pBluescript-SKII (+) (Stratagene) is PCR-amplified with primer P7 (5′-CGCCAGGGTTTCCAGTCACGAC-3′)(sequence number: 5) and primer P8 (5′-AGCGGATAACAATTTCACACAGGAAAC-3′)(sequence number: 6) as described. pCAGGS (ref. 7) was cleaved with EcoRI and blunt-ended with Klenow enzyme, and ligated with the PCR-amplified fragment, generating pCAGGS-P7.

(ii) Amplification of EYFP Gene

In this example, EYFP was obtained from EGFP (Genbank/EMBL accession number: U76561) by introducing six amino acid substitution

(Leu65Phe;Thr66Gly;Val69Leu;Gln70Lys;Ser73Ala;Thr204Tyr) by use of PCR-mediated mutagenesis. Then, full length cDNA of EYFP was obtained by using the EYFP gene as template, sense primer GFP-N2 (5′-GGATCCGGCATGGTGAGCAAGGGCGAGGAG-3′) (sequence number: 7), anti-sense primer GFP-N3 (5′-GGATCCGGTACCTCGAGCTTGTACAGCTCGTCCATG-3′) (sequence number: 8), and said Pfx.

Sense primer GFP-N2 consists of the underlined recognition sequence of BamHI, three-bases spacer, and the nucleotide sequence corresponding to amino acid 1 to 7 of EYFP. Antisense primer GFP-N3 consists of the underlined restriction sequences of BamHI, KpnI, and XhoI, and the complementary sequence corresponding to the carboxyl-terminus of ECFP (amino acid 233 to 239).

(iii) Amplification of ECFP Gene

In this example, ECFP was obtained from EGFP (Genbank/EMBL accession number: U76561) by introducing six amino acid substitution (Tyr67Trp; Asn147Ile; Met154Thr; Val164Ala) by use of PCR-mediated mutagenesis. Then, full length DNA of ECFP was obtained by using the EYFP gene as a template, sense primer XFPNot2 (5′- GCGGCCGCATGGTGAGCAAGGGCGAGGAGC -3′) (sequence number: 9), anti-sense primer XFP-Bgl (5′-AGATCTACAGCTCGTCCATGCCGAGAG -3′) (sequence number: 10), and said Pfx.

Sense primer XFPNot2 consists of the underlined recognition sequence of NotI and the nucleotide sequence corresponding to amino acid 1 to 8 of ECFP. Antisense primer XFP-Bgl consists of the underlined restriction site of BglII and the complementary sequence corresponding to the carboxyl-terminus of ECFP (amino acid 231 to 237).

(iv) Construction of pFret2

pCAGGS-P7 obtained in (i) was cleaved with XhoI and partially filled in with Klenow enzyme in the presence of dTTP and dCTP. EYFP obtained in (ii) was cleaved with BamHI and partially filled in with Klenow enzyme in the presence of dATP and dGTP. These two fragments were ligated by T4 DNA ligase. Then, the plasmid was cleaved with NotI and BglII and ligated with ECFP which was obtained in (iii) and cleaved with the same restriction enzymes. The obtained plasmid was named as pFret2.

(3) Construction of pRafras1722, an Expression Plasmid for the Monitoring Protein of Ras.

pFret2 as described in (2)-(iv) was cleaved with XhoI and NotI and ligated by using T4 DNA ligase with the chimeric gene described in (1)-(iii) cleaved with the same restriction enzymes, generating pRafras1722.

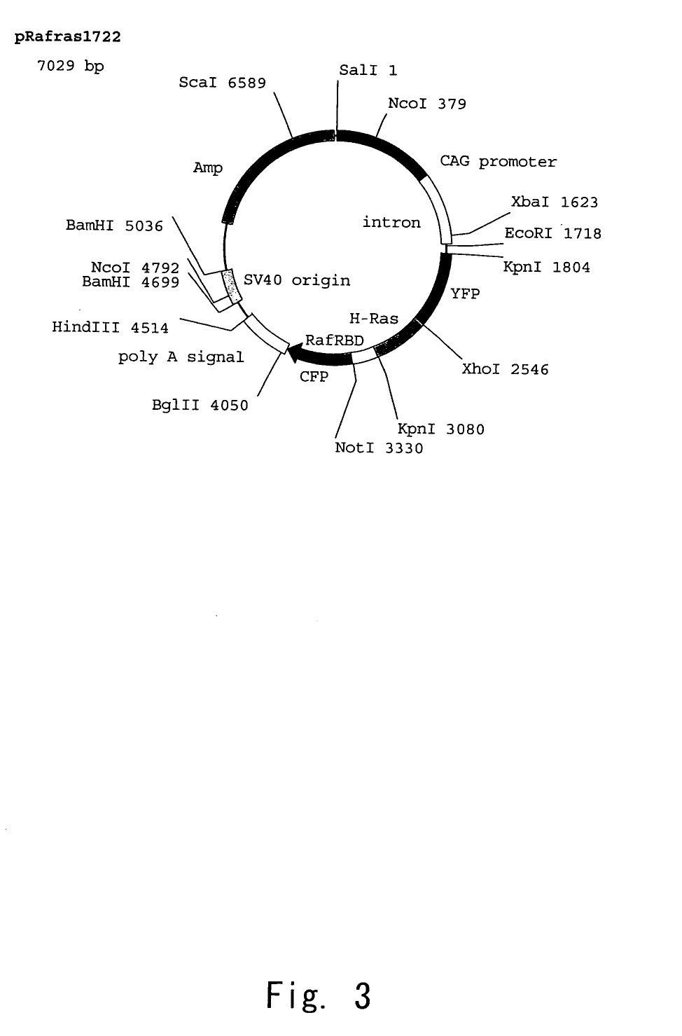

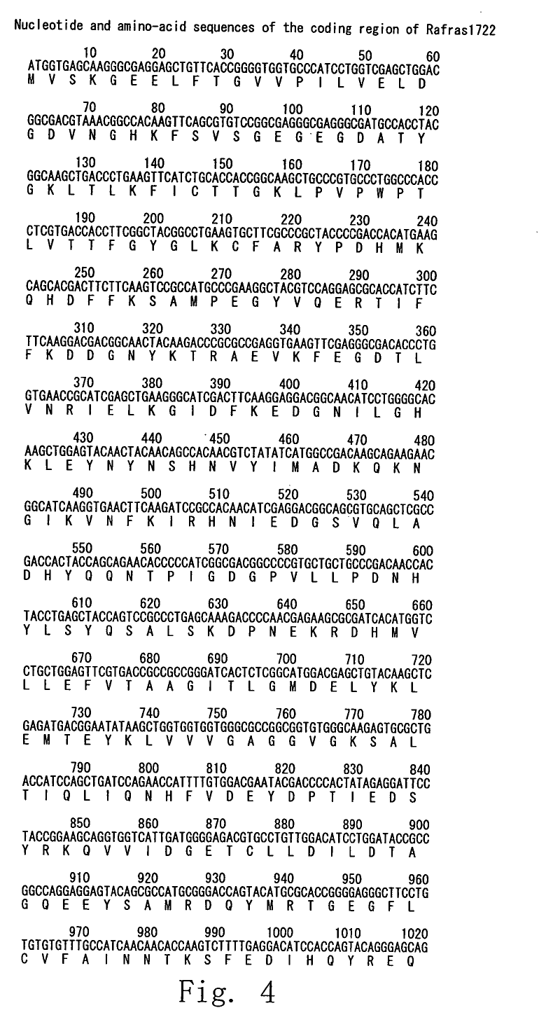

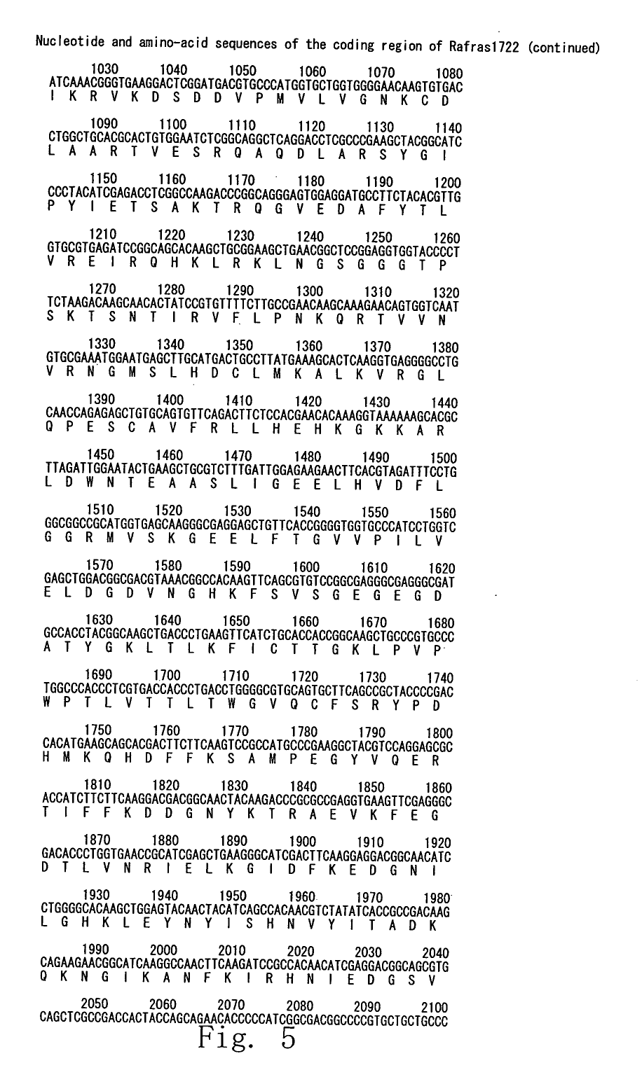

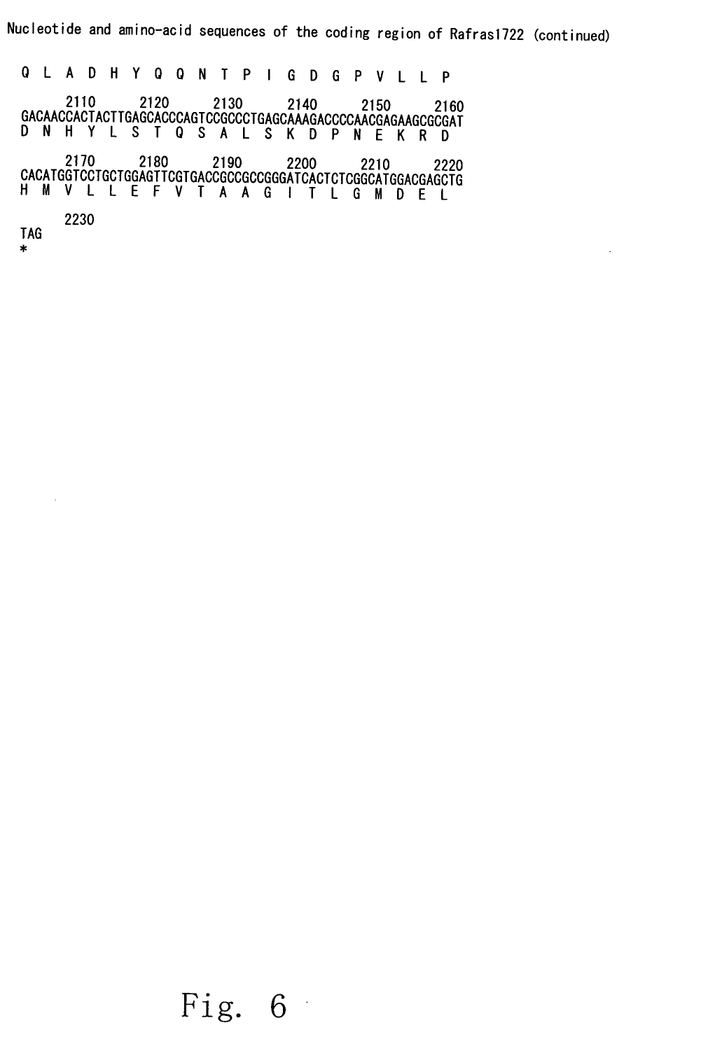

The structure and the nucleotide sequence of the coding region (sequence number: 11) and predicted amino acid sequence (sequence number: 12) are shown in FIG. 3 and FIGS. 4 to 6, respectively. Detailed explanation is as follows.

| | | |
|---|---|---|
| nt | 1-717: | Aequorea EYFP |
| nt | 718-723: | Linker |
| nt | 724-1239: | Ras |
| nt | 1240-1257: | Linker |
| nt | 1258-1500: | Raf |
| nt | 1504-1509: | Linker |
| nt | 1510-2220: | Aequorea ECFP |

(4) Expression of Ras Monitoring Protein Rafras1722 in Mammalian Cells and its Spectrum Analysis

HEK293T cells derived from human embryonic kidney cells were cultured in DMEM (Nissui) containing 10% fetal calf serum. pRafras1722 described in (3) and an expression vector of guanine nucleotide exchange factor Sos (pCAGGS-mSos) or an expression vector of GTPase activating protein Gaplm (pCAGGS-mSos) were transfected into HEK293T cells by calcium coprecipitation method. After transfection, HEK293T cells were further cultured in DMEM containing 10% FBS to allow the Ras monitoring protein expressed. Forty-eight hours later, cells were washed with phosphate-buffered saline and lysed in lysis buffer (20 mM Tris-HCl, pH 7.5, 150 mM NaCl, 5 mM MgCl2, 0.1% Triton X-100). The cell lysates were centrifuged at 10,000×g and the supernatant was collected.

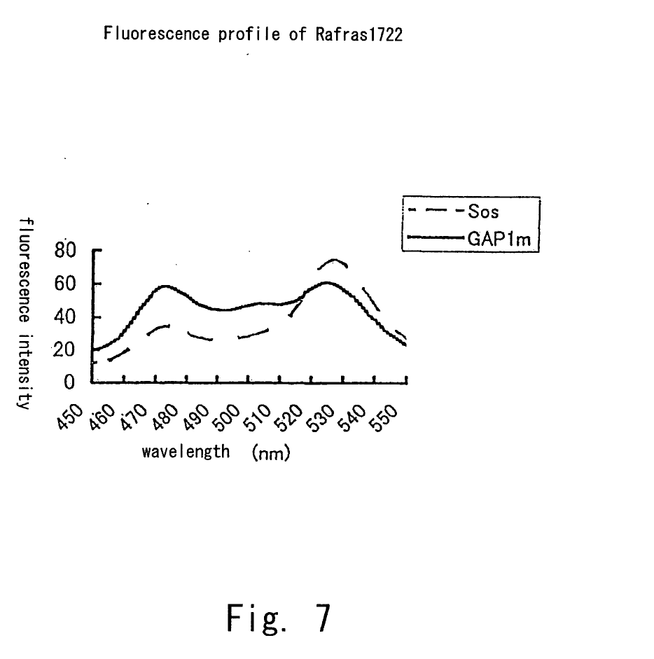

The supernatant was transferred into 1 ml cuvette of fluorescent spectrometer (Nippon Bunko, FP-750) and fluorescent intensity was analyzed from 450 nm to 550 nm at an excitation wavelength of 433 nm. The obtained fluorescent profile is shown in FIG. 7.

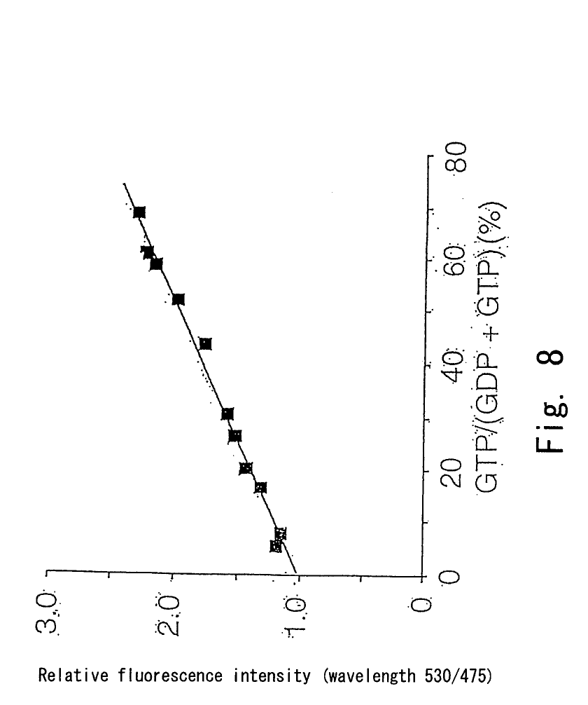

The transfected HEK293T cells were also labeled with 32Pi and lysed. Ras monitoring protein was immunoprecipitated with anti-GFP antibody and bound GTP and GDP were separated by thin layer chromatography. By this method, the FRET efficiency ((fluorescent intensity at 530 nm)/ (fluorescent intensity at 475 nm) at an excitation wavelength of 433 nm) can be correlated with the actual GTP/GDP ratio (FIG. 8). In the FIG. 8, FRET efficiency and GTP-binding are shown as (fluorescent intensity (530 nm/475 nm)) and (GTP/(GDP+GTP) (%)), respectively.

(5) Expression of Ras Monitoring Protein in Mammalian Cells and Analysis with Time-Lapse Fluorescent Microscope.

COS7 cells derived from monkey kidney cells were cultured in phenol-red-free MEM (Nissui) containing 10% fetal calf serum. pRafras1722 described in (3) was transfected into COS7 cells by calcium phosphate method. After transfection, COS7 cells were cultured in phenol-red-free MEM (Nissui) containing 10% fetal calf serum to allow the expression of Ras monitoring protein. Forty-eight hours after transfection, cells were observed with time-lapse fluorescent microscope.

Cell images were obtained with an imaging system consisting of Metamorph image analyzing software (Roper Scientific Japan) and inverted fluorescent microscope Axiovert 100 (Carl Zeiss) equipped with Xenon lamp, revolving filter changers for excitation filters and emission filters (LUDL electronic), and high sensitivity cooled CCD camera Micromax 450 (Photometrix). Excitation filters, emission filters and dichroic mirrors were obtained from Omega.

Cells were illuminated with an excitation wavelength of 430 nm and fluorescent images of ECFP donor protein at 475 nm and fluorescent images of EYFP acceptor protein at 530 nm were acquired. After data acquisition, each pixel of the digital images was assigned from blue to red colors, depending on the levels of EYFP/ECFP fluorescent ratios.

### Example 2

### Establishment of Cell Lines for the Measurement of Ras Activation

Mouse fibroblast NIH3T3 cells were cultured in DMEM (Nissui) containing 10% fetal calf serum. pRafras1722 and pSV2neo (Genbank/EMBL: U02434) were co-transfected into NIH3T3 cells with FuGene6 (Roche). Forty-eight hours after transfection, cells were replated at 1:10 and cultured in said DMEM containing 0.5 mg/ml G418 (Gibco-BRL). Medium was replaced every three days. After 2 weeks, cells of well-isolated colonies were cloned and named as 3T3-Rafras cells.

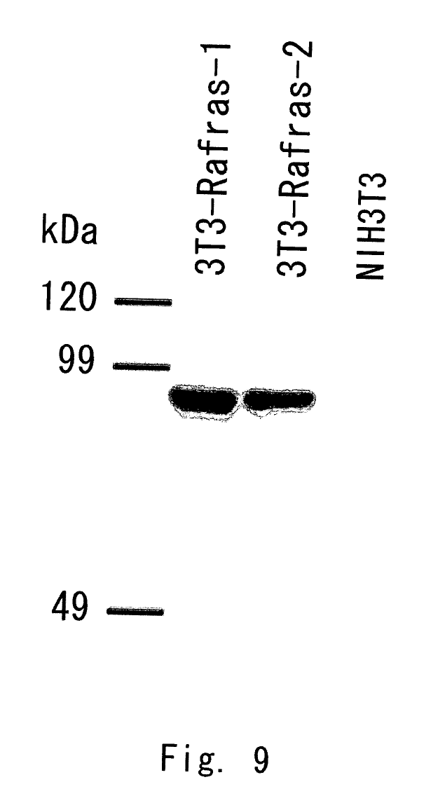

The 3T3-Rafras cells were cultured in DMEM containing 10% fetal calf serum and 0.5 mg/ml G418 to allow the expression of monitoring protein for Ras activity. Then, the expression of said protein was examined by anti-Ras antibody (Transduction Lab) by immunoblotting. Expression of expected ca. 80 kDa protein was confirmed (FIG. 9).

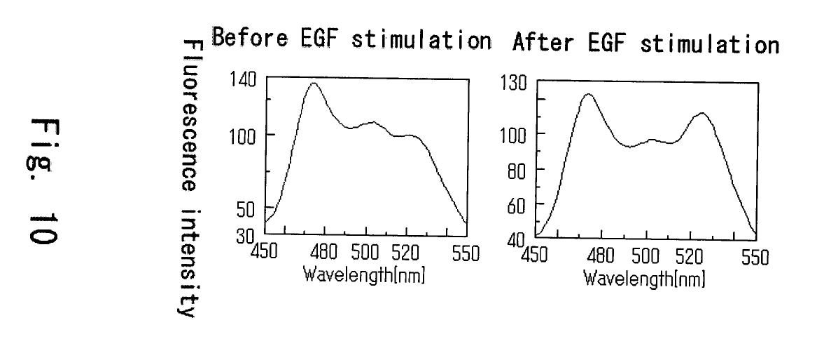

Furthermore, the cells were stimulated with epidermal growth factor (EGF) (Sigma), and the FRET efficiency before and after EGF stimulation was analyzed as described in example 1. The fluorescence profile before and after EGF stimulation is shown in FIG. 10.

### Example 3

### Measurement of Rap1A Activity by Using RAI-chu311

(1) Construction of a Gene Encoding a Chimera of Rap1A and RalGDS

(i) Amplification of Rap1A Gene

Using Rap1A cDNA (Genbank/EMBL accession number: X12533) as a template, sense primer hRap1Xh (5′-GGCTCGAGATGCGTGAGTACAAGCTAGTGG—3″)(sequence number: 13), anti-sense primer Rap172RalGDS (5′-GCGGATGATACAGCAGTCGCCACCTCCGGATCCGCCGGTACC TCCACCACCGGTTCCACCTCCGGAGCCATTGATCTTTGACTTTG CAGAAG-3′) (sequence number: 14), and heat stable DNA polymerase Pfx (Gibco-BRL, Bethesda, U.S.A.), DNA corresponding to the amino acid 1 to 172 of Rap1A was amplified by polymerase chain reaction (PCR).

Sense primer hRap1Xh consists of the underlined recognition sequence of restriction enzyme XhoI and the DNA sequence corresponding to the amino acid 1 to 8 of Rap1A. Meanwhile, anti-sense primer Rap172RalGDS consists of a complementary DNA sequence of RalGDS (Genbank/EMBL accession number: U14417) corresponding to the amino-terminal region of the Rap1 binding domain (amino acid 211 to 217), spacer sequence (underlined), and DNA sequence of Rap1A corresponding to amino acid166 to 172.

(ii) Amplification of RalGDS Gene

Using RalGDS cDNA (Genbank/EMBL accession number: U14417) as a template, sense primer RalGDS-F (5′-GGCGACTGCTGTATCATCCGC -3″)(sequence number: 15), anti-sense primer RalGDSR (5′-CGCGGCCGCCCCGCTTCTTGAGGACAAAGTC -3′) (sequence number: 16), and said Pfx, DNA corresponding to amino acid 51 to 131 of Raf were amplified by polymerase chain reaction (PCR).

Sense primer RalGDS-F consists of a DNA sequence corresponding to the amino acid 211 to 217 of RalGDS. Meanwhile, anti-sense primer RalGDSR consists of the underlined Not restriction sequence and the complementary DNA sequence of RalGDS corresponding to carboxyl-terminal region of the Rap1 binding domain (amino acid 291 to 297).

Using a mixture of the amplified DNAs described in (i) and (ii) as templates, sense primer hRap1Xh, anti-sense primer RalGDSR, and said Pfx, DNA of a chimera of Rap1 and RalGDS was amplified by PCR. Then, the obtained DNA fragment was cloned into pCR-bluntII-TOPO (Invitrogen), followed by transformation of E. coli. Then, the E. coli was cultured and plasmids were prepared by SDS-alkaline method.

(2) Construction of pRai-chu311, an Expression Plasmid for the Monitoring Protein of Rap1.

In example 1-(2)-(ii), antisense primer GFP-dllR (5′-GGATCCGGTACCTCGAGGGCGGCGGTCACGAACTCCAGCAG-3′)(sequence number: 17) was used instead of the primer GFP-N3 to obtain a cDNA of EYFP that lacks eleven amino acids of the carboxyl terminus. This truncated EYFP cDNA was replaced with the corresponding region of pFRET2. This vector was cleaved with XhoI and NotI, and ligated with the chimeric gene described in (1)-(ii) cleaved with the same restriction enzymes by using T4 DNA ligase, generating pRai-chu311.

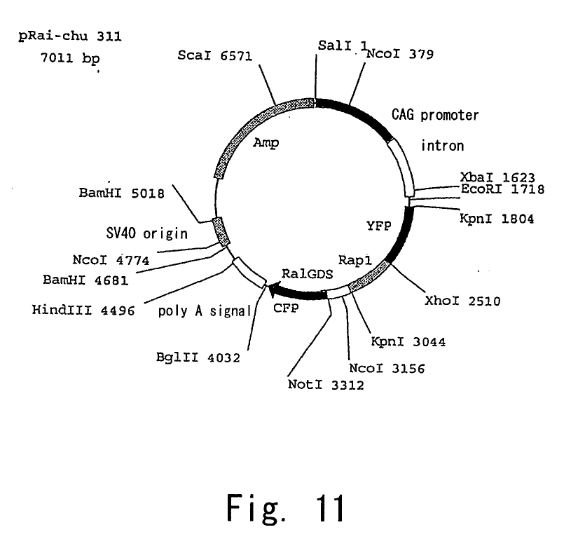

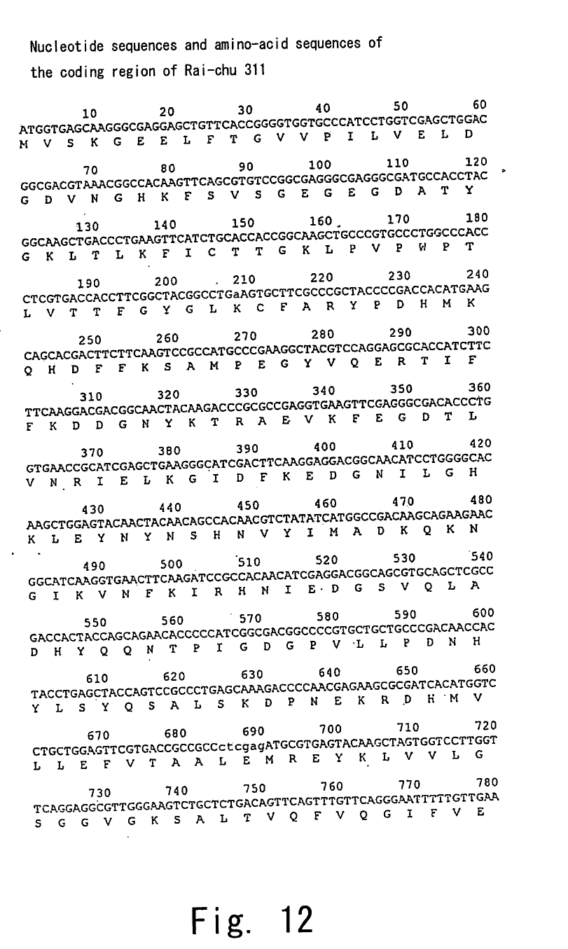

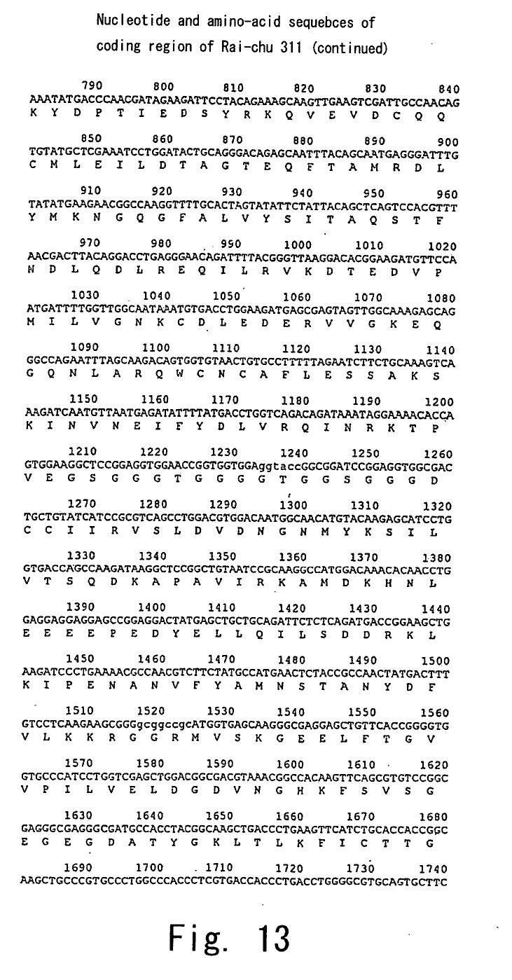

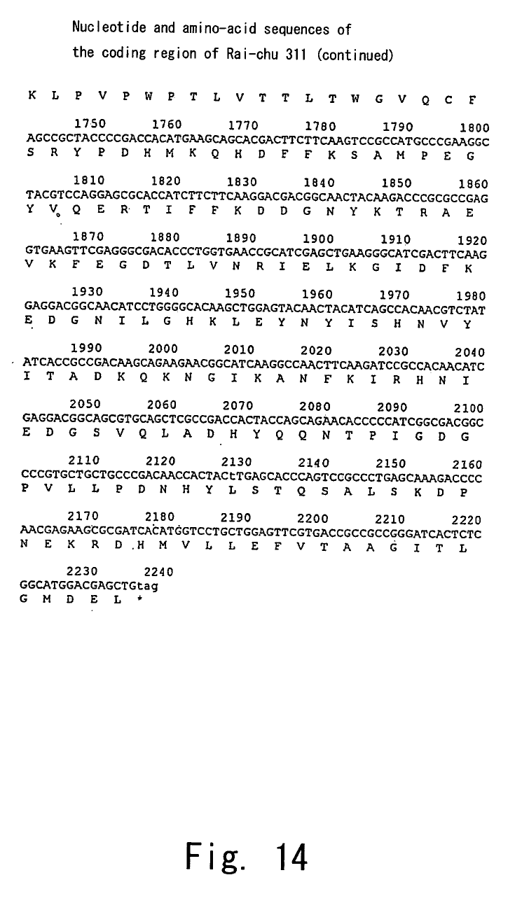

The structure and the nucleotide sequence of the coding region (sequence number: 18) and predicted amino acid sequence (sequence number: 19) are shown in FIG. 11 and FIGS. 12 to 14, respectively. Detailed explanation is as follows.

| | | |
|---|---|---|
| nt | 1-684: | Aequorea EYFP |
| nt | 685-690: | Linker |
| nt | 691-1206: | Rap1 |
| nt | 1207-1257: | Linker |
| nt | 1258-1515: | RalGDS |
| nt | 1516-1521: | Linker |
| nt | 1522-2235: | Aequorea ECFP |

(3) Expression of Rap1A Monitoring Protein Rai-chu311 in Mammalian Cells and its Spectra Analysis

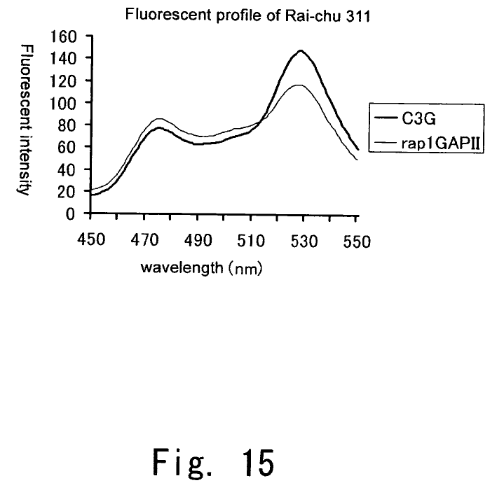

Analysis was performed as in example 1-(4). The fluorescent profile is shown in FIG. 15.

### Example 4

### Measurement of R-Ras Activity by Using Rai-chu158

(1) Construction of pRai-chu158

(i) Amplification of R-Ras gene

Using R-Ras cDNA (Genbank/EMBL accession number: M14948, M14949) as a template, sense primer RRas28F (5′-CCCCTCGAGACACACAAGCTGGTGGTC -3″)(sequence number: 20), anti-sense primer RRas2O4R (5′-GCCGGTACCGCCACTGGGAGGGCTCGGTGGGAG -3′) (sequence number: 21), and heat stable DNA polymerase Pfx (Gibco-BRL, Bethesda, U.S.A.), DNA corresponding to amino acid 28 to 204 of R-Ras was amplified by polymerase chain reaction (PCR). Sense primer RRas28F consists of the underlined XhoI restriction sequence and the DNA sequence corresponding to the amino acid 28 to 33 of R-Ras. Anti-sense primer RRas204R consists of the underlined KpnI restriction sequence and the complementary DNA sequence corresponding to the carboxyl-terminal region of R-Ras (amino acid 198 to 204).

(ii) Preparation of the Restriction Fragment.

The PCR product obtained in (i) was cleaved with XhoI and KpnI.

(iii) Construction of pRai-chu158, an Expression Plasmid for R-Ras Activity Monitoring Protein.

pRafras1722 obtained in example 1 was cleaved with XhoI and KpnI to obtain a DNA fragment that lacks the Ras gene. This plasmid was ligated with the restriction fragment obtained in (ii), generating pRai-chu158.

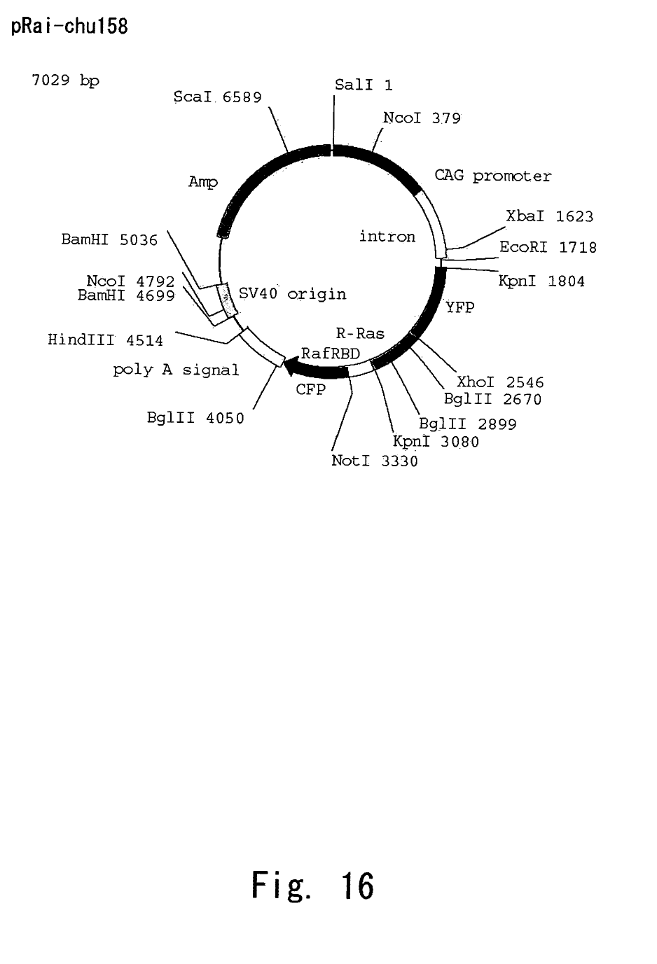

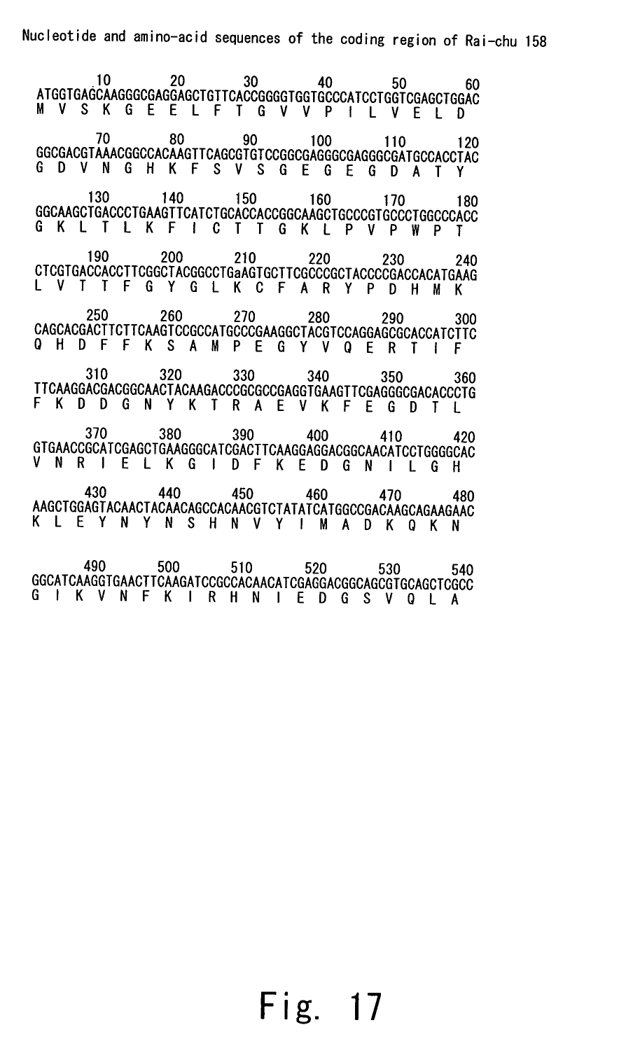

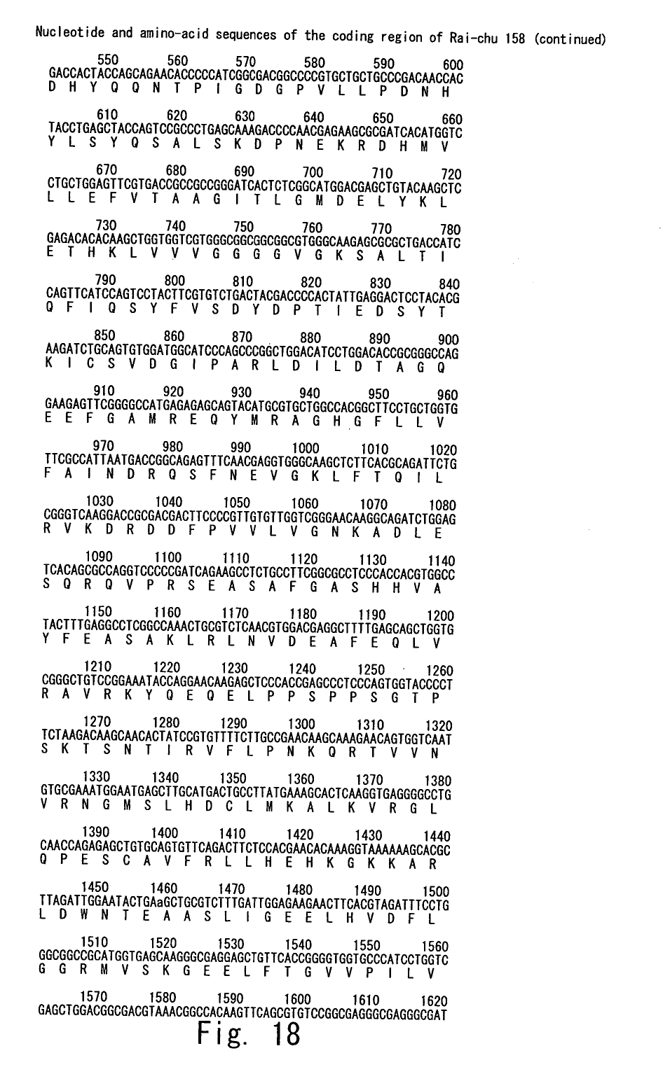

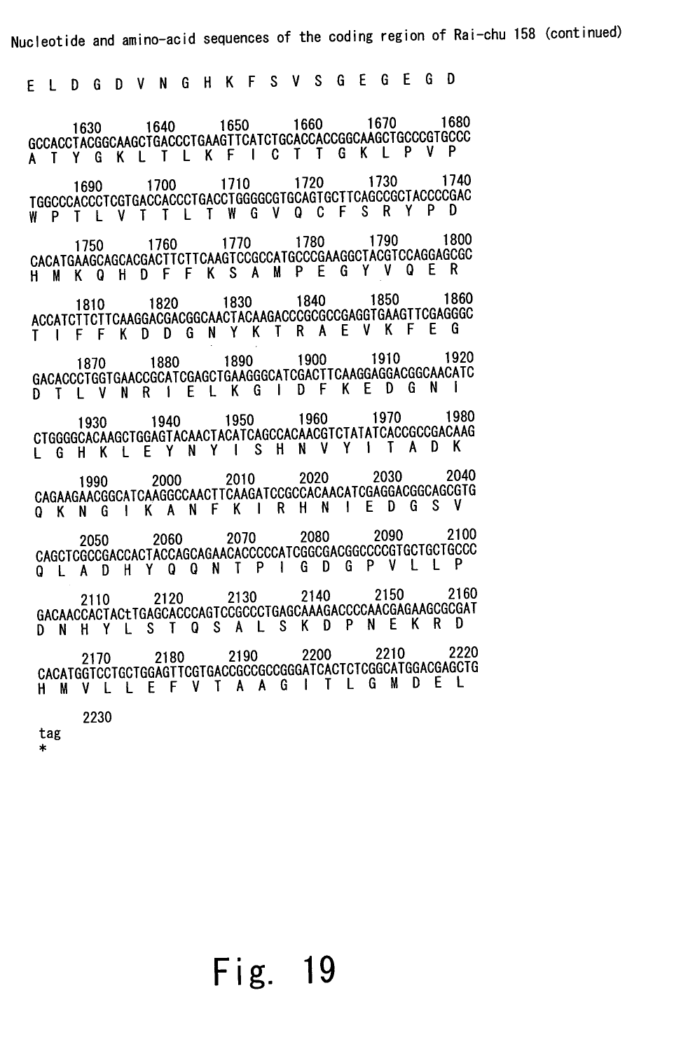

The structure and the nucleotide sequence of the coding region (sequence number: 22) and predicted amino acid sequence (sequence number: 23) are shown in FIG. 16 and FIGS. 17 to 19, respectively. Detailed explanation is as follows.

| | | |
|---|---|---|
| nt | 1-717: | Aequorea EYFP |
| nt | 718-723: | Linker |
| nt | 724-1251: | R-Ras |
| nt | 1252-1257: | Linker |
| nt | 1258-1500: | Raf |
| nt | 1501-1509: | Linker |
| nt | 1510-2220: | Aequorea ECFP |

(3) Expression of R-RasA Monitoring Protein Rai-chu158 in Mammalian Cells and its Spectra Analysis

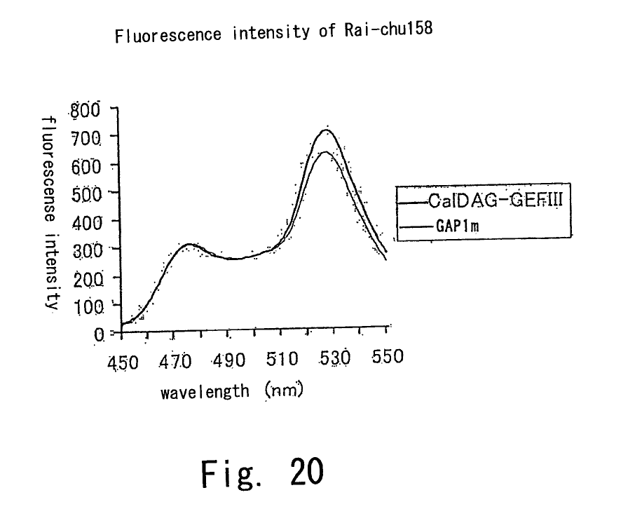

Analysis was performed as in the example 1 (4). The fluorescent profile is shown in FIG. 20.

### Example 5

### Construction of a Gene Encoding a Monitoring Protein Which Carries a Temperature-Sensitive Mutation in the Effector-Binding Domain of Ras

(1) Construction of pRai-chu119

(i) Amplification of a Mutated Ras Gene

Using the cDNA used in example 1, sense primer hRasXh (described in example 1), anti-sense primer RasI38LR (5′-GGAATCCTCTAGAGTGGGGTCG -3′) (sequence number: 24), and the DNA polymerase Pfx, DNA corresponding to amino acid 1 to 39 of Ras was amplified by polymerase chain reaction (PCR).

Antisense primer RasI38LR consists of a DNA sequence corresponding to the amino acid 35 to 42 of Ras wherein a codon for Ile is substituted for Leu as indicated by underline. This point mutation is known to generate a temperature sensitive Ras mutant (ref. 8).

Similarly, using the cDNA used in example 1, sense primer RasI36LF (5′-CGACCCCACTCTAGAGGATTCC-3′)(sequence number 25), anti-sense primer Ras172Raf (described in example 1), and the DNA polymerase Pfx, DNA corresponding to amino acid 32 to 172 of Ras was amplified by polymerase chain reaction (PCR). By using the mixture of these two amplified DNA fragments as a template, sense primer hRasXh, and anti-sense primer Ras172Raf, a DNA fragment corresponding to amino acid 1 to 172 of Ras that contains a point mutation of Ile36Leu was amplified by PCR.

(ii) Preparation of Restriction Fragment.

The PCR product obtained in (i) was cleaved with XhoI and KpnI.

(iii) Construction of pRai-chu119, an Expression Plasmid for R-Ras Activity Monitoring Protein.

pRafras1722 obtained in example 1 was cleaved with XhoI and KpnI to obtain a DNA fragment that lacks the Ras gene. This plasmid was ligated with the restriction fragment obtained in (ii), generating pRai-chu119.

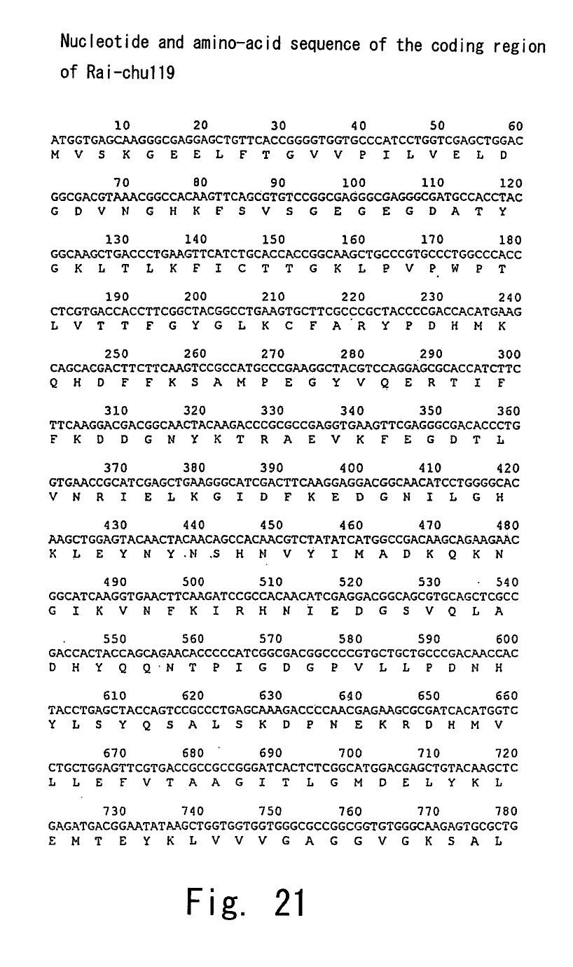

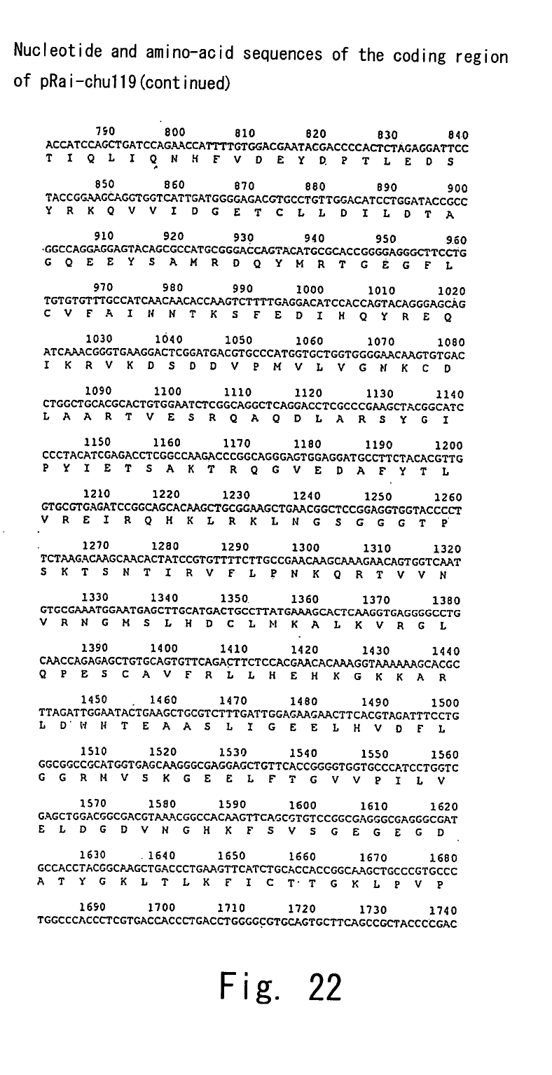

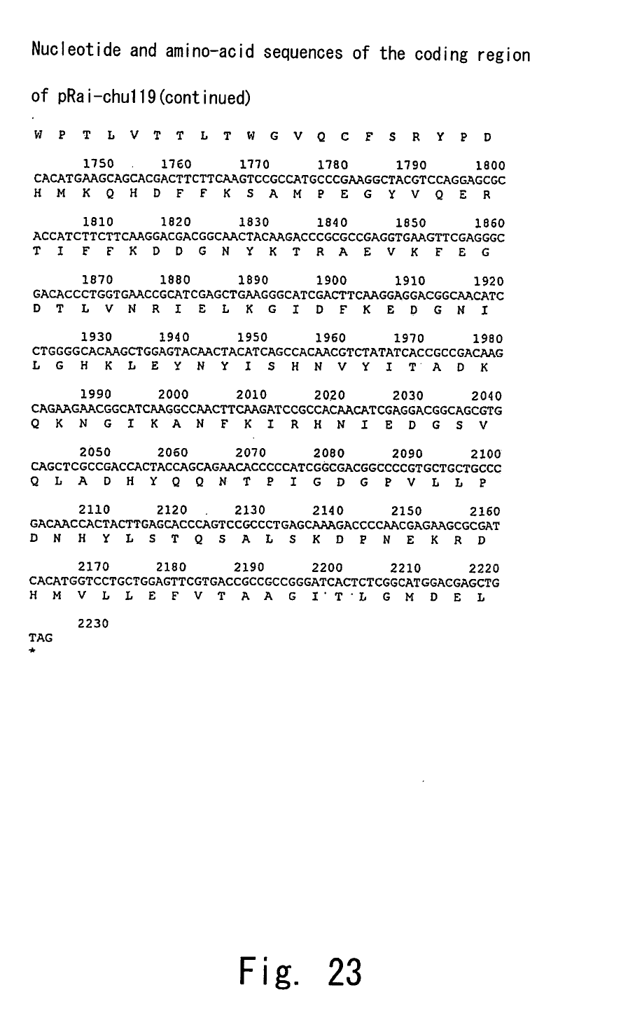

The structure and the nucleotide sequence of the coding region (sequence number: 26) and predicted amino acid sequence (sequence number: 27) are shown in FIGS. 21 to 23.

(2) Expression of Ras Monitoring Protein Rai-chu119 in Mammalian Cells and its Spectrum Analysis

HEK293T cells derived from human embryonic kidney cells were cultured in DMEM (Nissui) containing 10% fetal calf serum. pRafras1722 described in (3) or pRai-chu119 with an expression vector of guanine nucleotide exchange factor Sos (pCAGGS-mSos) were transfected into HEK293T cells by calcium coprecipitation method. After transfection, HEK293T cells were further cultured in DMEM containing 10% FBS to allow the Ras monitoring protein being expressed. Twenty-four hours after transfection, temperature of the incubator was changed to 33 or 40° C. After further 24 hrs, cells were washed with phosphate-buffered saline and lysed in lysis buffer (20 mM Tris-HCl, pH 7.5, 150 mM NaCl, 5 mM MgCl2, 0.1% Triton X-100). The cell lysates were centrifuged at 10,000×g and the supernatant was collected.

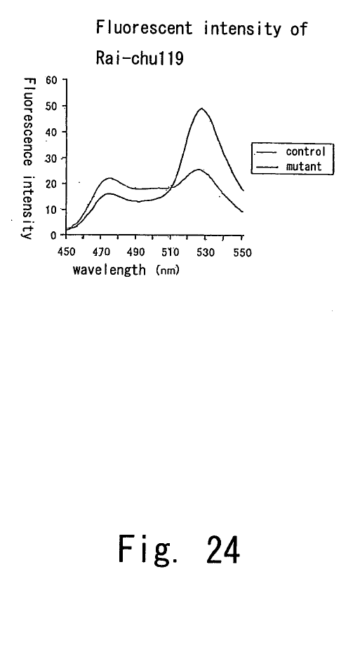

The supernatant was transferred into 1 ml cuvette of fluorescent spectrometer (Nippon Bunko, FP-750) and fluorescent intensity was analyzed from 450 nm to 550 nm at an excitation wavelength of 433 nm. The obtained fluorescent profile is shown in FIG. 24.

### Example 6

### Generation of Transgenic Mice Expressing Rafras1722 and the Measurement of Ras Activation Using Cardiac Myocytes

(1) pRafras1722 described in example 1 was cleaved with restriction enzyme SpeI and BamHI, and subjected to agarose electrophoresis to obtain 4.5 kb DNA fragment containing promoter, intron, coding region, and poly A signal. The DNA fragment was electro-eluted from the gel and purified with Qiagen-tip 20 (Qiagen). The DNA was injected to mouse fertilized egg (DBF1, SLC Co.) and transferred to oviduct of ICR mouse. From the offspring, tail DNA was obtained by Proteinase K treatment, phenol-chloroform extraction, and isopropanol precipitation. DNA was dissolved in double distilled water at 37° C.

(2) By using the mouse cDNAs as templates, sense primer RafRBDx (5′-CTCGAGCCTTCTAAGACAAGCAACACT-3′) (sequence number: 28), and anti-sense primer XFPNseq (5′-CGTCGCCGTCCAGCTCGACCAG -3′) (sequence number: 29), PCR was performed. With these primers, a DNA fragment corresponding to the junction of Raf and ECFP in Rafras1722 gene was amplified. Appearance of the expected 314 bp fragment indicates the integration the Rafras1722 gene into the mouse genome. Among 35 newborn mice, 7 were positive.

(3) The PCR-positive F1 mice were mated with C57/Black mice (SLC, Hamamatsu, Japan). From the newborn F2 mice, cardiac ventricle was excised and cut into pieces. By the addition of PBS containing 0.05% trypsine and 0.5 mM EDTA for 10 min at 37° C., cardiac myocytes were harvested. By repeating this procedure for 6 times, myocytes were collected. Then, after the addition of DMEM containing 10% FBS and low-speed centrifugation, myocytes were plated onto dishes.

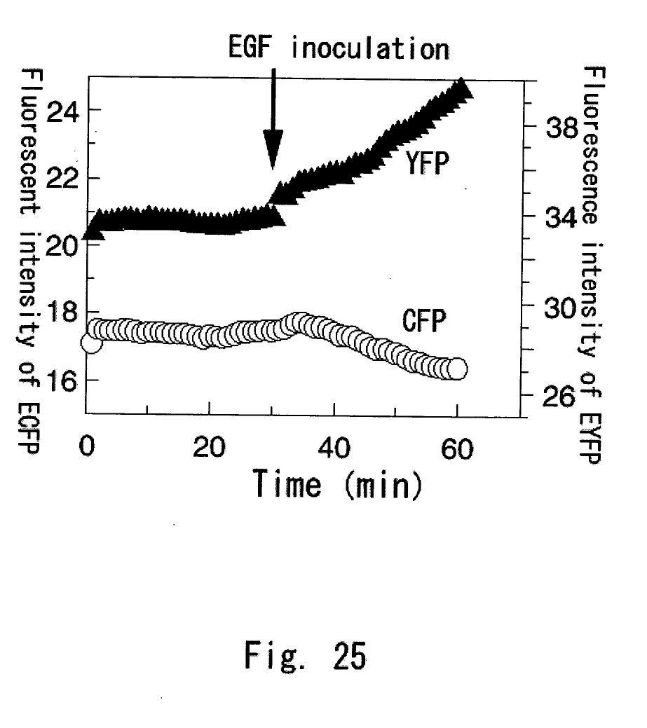

(4) The obtained myocytes were replated on glass base dishes. After 6 hours incubation in serum-free DMEM, EGF was added and cells were observed as described in example 1- (5). The time-course of fluorescent intensities of ECFP and EYFP upon EGF stimulation is shown in FIG. 25. The EGF-induced activation of Ras was observed in primary cardiac myocytes derived from transgenic mice.

### Example 7

### Specificity of Rafras1722 to Guanine Nucleotide Exchange Factors and GTPase Activating Proteins

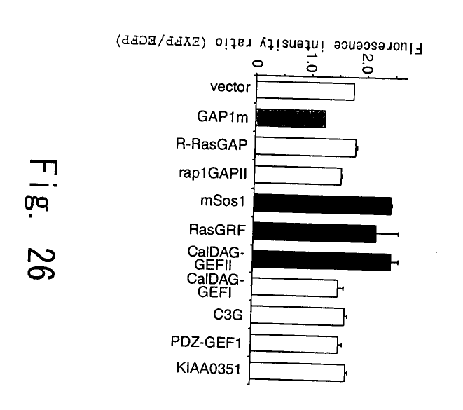

By the same procedure shown in example 1- (4), specificity of Rafras1722 to guanine nucleotide exchange factors and GTPase activating proteins was examined. Expression vectors for GAPlm, R-RasGAP, rapGAPII, mSosI, RasGRF, CalDAG-GEF1, C3G, PDZ-GEF1, and KIAA0351 were used for GTPase activating protein or guanine nucleotide exchange factors. As shown in FIG. 26, the FRET efficiency was decreased by GAPlm, a Ras GAP, but not by R-RasGAP or Rap1GAPII, GAPs for R-Ras or Rap1. In contrast, the FRET efficiency was increased by GEFs for Ras including mSosl, RasGRF, and CalDAG-GEFII, but not by GEFs for the other Ras-family G proteins including CalDAG-GEF1, C3G, PDZ-GEF1, and KIAA0351. These observations indicate that the FRET efficiency of Rafras1722 is regulated by the guanine nucleotide exchange factors and GTPase activating proteins for the authentic Ras.

### Example 8

### Construction of a Rap Monitoring Protein Rai-chu404 and its Specificity to Guanine Nucleotide Exchange Factors and GTPase Activating Proteins

(1) Construction of a Gene Encoding Rai-chu404, a Chimera of Rap1A and Raf

By PCR mutagenesis, seven amino-acid substitutions (Thr66Gly; Val69Leu; Ser73Ala; Met154Thr; Val164Ala; Ser176Gly; Thr204Tyr) were introduced into EGFP (Genbank/EMBL accession number: U76561), which was substituted for EYFP of Rai-chu311 described in example 3. Then, the KpnI/NotI fragment encoding RalGDS in this modified Rai-chu311 was replaced with the KpnI/NotI fragment of Rafras1722 encoding Raf, generating pRai-chu404. The nucleotide sequence and predicted amino-acid sequence of the coding region are shown in sequence number 30 and 31, respectively.

(2) The effect of guanine nucleotide exchange factors on the FRET efficiency of Rai-chu404 was examined as described in example 7.

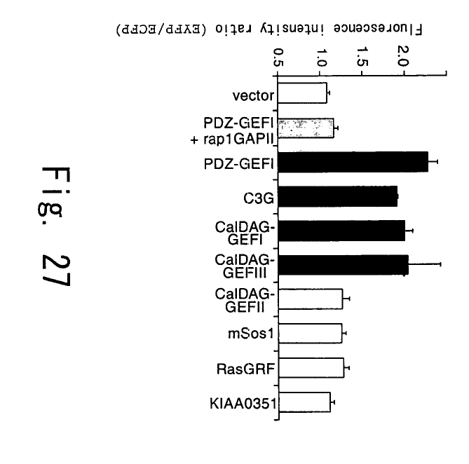

PDZ-GEF1, C3G, CalDAG-GEFI, CalDAG-GEFIII were used as guanine nucleotide exchange factors for Rap1. As controls, guanine nucleotide exchange factors for Ras including CalDAG-GEFII, mSosland RasGRF and a guanine nucleotide exchange factor for Ral, KIAA0351, was used as described in reference 9. As shown in FIG. 27, only the guanine nucleotide exchange factors for Rap1 could increase the FRET efficiency of Rai-chu404.

### Example 9

### Construction of Rai-chu101X and Rai-chu404X Monitoring Proteins that Contain the CAAX Box of K-Ras

In the experimental protocol shown in example 1-(1)-(i), a primer with XbaI restriction site (sequence number 32) was used instead of the forward primer XFP-Bgl. The amplified ECFP gene was ligated to the CAAX region of K-Ras gene as described in reference 11. The fused ECFP-CAAX gene was substituted for the ECFP gene of Rafras1722 or Rai-chu404. The resulting vectors were designated as pRai-chu101X and pRai-chu404X. The nucleotide sequences of the coding regions (sequence number: 33 and 35) and predicted amino acid sequences (sequence number: 34 and 36) are shown.

### Example 10

### Visualization of the Activity of Ras and Rap1 in COS1 Cells Expressing Rai-chu101X and Rai-chu404X

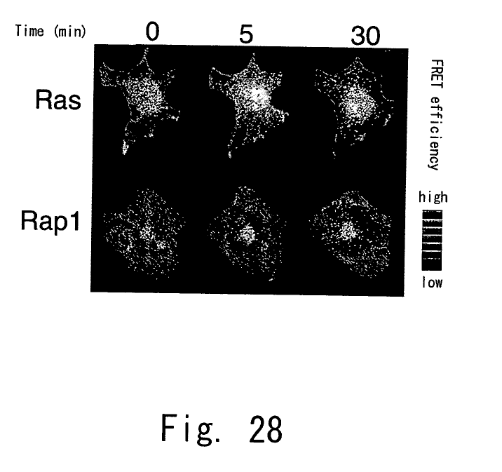

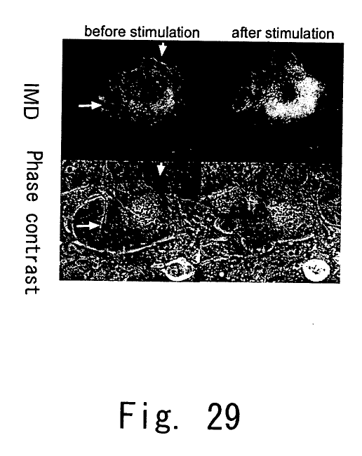

COS1 cells transfected with pRai-chu101X or pRai-chu404X described above were cultured for 24 hrs. Cell images were obtained with an imaging system described in the example 1-(5). The time-course of fluorescence intensities of ECFP and EYFP and fluorescence ratio (EYFP/ECFP) in the cells stimulated with EGF are shown in FIG. 28. This figure is displayed by the IMD mode wherein the regions of high fluorescence ratio are shown in red, those of low fluorescence ratio are shown in blue, and the intensity reflects that of ECFP. By the stimulation of EGF, the fluorescence ratio, which reflects the FRET efficiency, gradually increases from the periphery to the center in the cells expressing Rai-chu101X. Meanwhile, the activity increases from the center to the periphery in the cells expressing Rai-chu404X. When similar experiments were performed with semiconfluent cells, it became clear that Ras could not be activated where cells were in contact with neighboring cells and that Ras was activated only from the free edges (FIG. 29). Thus, the invented monitoring proteins enables us to obtain the spatio-temporal information on the activity of Ras-family G proteins.

### Example 11

### Visualization of the Activation of Ras and Rap1 in PC12 Cells Expressing Rai-chu101X and Rai-chu404X

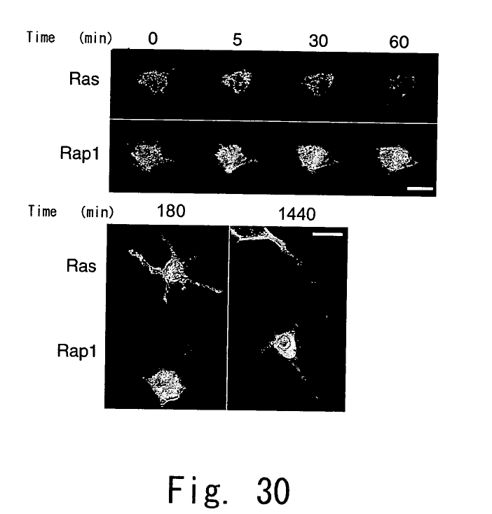

PC12 cells transfected with pRai-chu101X or pRai-chu404X described above were cultured for 24 hrs. Cell images were obtained with an imaging system described in the exampole 1 (5). The time-course of fluorescence intentisities of ECFP and EYFP and fluorescence ratio (EYFP/ECFP) in the cells stimulated with nerve growth factor are shown in FIG. 30. This figure is displayed by IMD mode wherein the regions of high fluorescence ratio are shown in red, those of low fluorescence ration are shown in blue, and the intensity reflects that of ECFP. During the induction of neuronal differentiation of PC12 cells, Ras was activated from the periphery of the cell body in the induction phase, whereas after differentiation the Ras activity, which is known to be essential for the survival of the cells, were maintained only at the extended neurites. Namely, it became evident that the activation of Ras occurs at different intracellular regions during the stages of differentiation. In contrast to Ras, Rap1 is activated from the perinuclear region and suppressed at the neurites. This observation indicates that the Ras-family G proteins are regulated differently at the different intracellular localization.

### Example 12

### Construction of a Monitoring Protein for Rac1, Raichu-101X

(1) Construction of a Chimeric Gene Between Rac1 and Pak1.

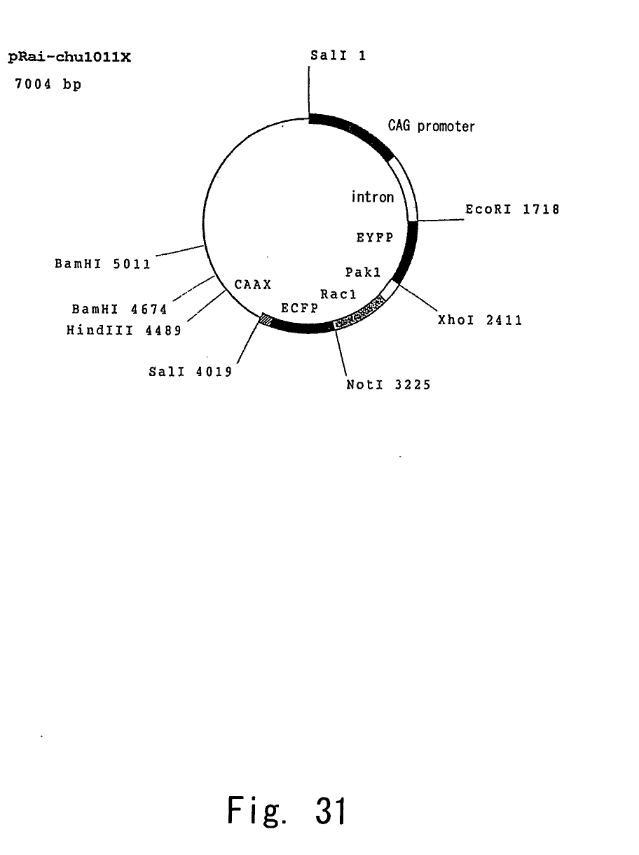

By using the PCR-based procedure described in the examples 1 and 9 pRai-chu1011X was obtained with the cDNAs of Rac1(Genbank/EMBL accession number M29870) and Pak1 (Genbank/EMBL accession number NM002576) used as templates. The structure (FIG. 31) and the nucleotide sequence of the coding region (sequence number: 37) and predicted amino acid sequence (sequence number: 38) are shown.

Detailed Explanation is as Follows.

| | | |
|---|---|---|
| nt | 1-684: | Aequorea EYFP |
| nt | 685-690: | linker |
| nt | 691-939: | Pak1 |
| nt | 940-969: | linker |
| nt | 970-1497: | Rac1 |
| nt | 1498-1506: | linker |
| nt | 1507-2217: | Aequorea ECFP |
| nt | 2218-2229: | linker |
| nt | 2230-2289: | carboxyl-terminal region of K-Ras (CAAX box) |

(2) Construction of Mutants of the Chimeric Gene

By the PCR-mediated mutagenesis, thymine was substituted for guanine at position 1004 of pRai-chu1011X (sequence number 37), whereby Val was substituted for Gly in the predicted amino acid sequence, generating pRai-chu1012X. Similarly, adenine was substituted for cytosine at position 1019, whereby Asn was substituted for Thr in the predicted amino acid sequence, generating pRai-chu1013X. In the mutant protein Rai-chu1012X, the GTPase activity is decreased, rendering this mutant constitutively active. Meanwhile, in the mutant protein Rai-chu1013X, the binding of Rac1 to GTP is decreased, rendering this protein inactive.

(3) Expression of Rac1 Monitoring Proteins in Mammalian Cells and Their Analysis by Spectrometer.

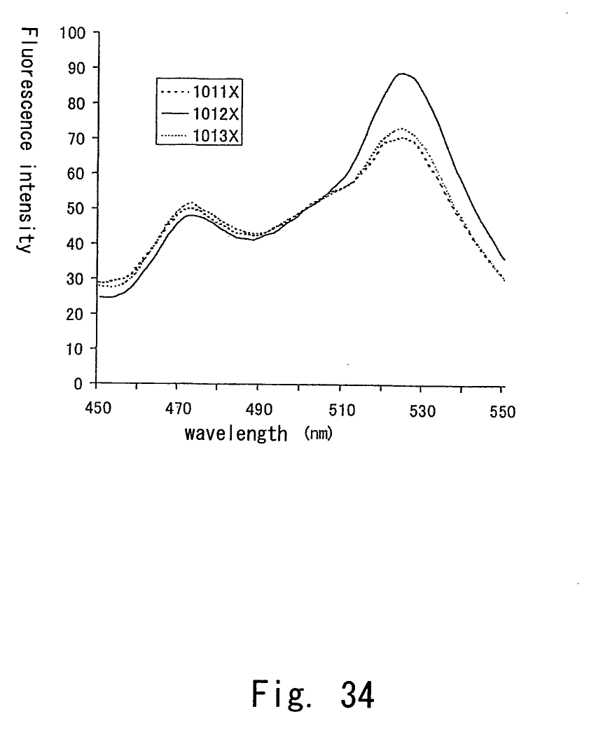

Rai-chu1011X, Rai-chu1012X, and Rai-chu1013X were expressed in the cells and analyzed by the method described in the example 1-(4). The obtained profiles of spectrum are shown in FIG. 34. The FRET efficiency of Rai-chu1013Xis lower than those of the wild-type Rai-chu1011X and the active form Rai-chu1012X.

### Example 13

### Construction of a Monitoring Protein for Cdc42, Raichu-1054X

(1) Construction of a Chimeric Gene Between Cdc42 and Pak1.

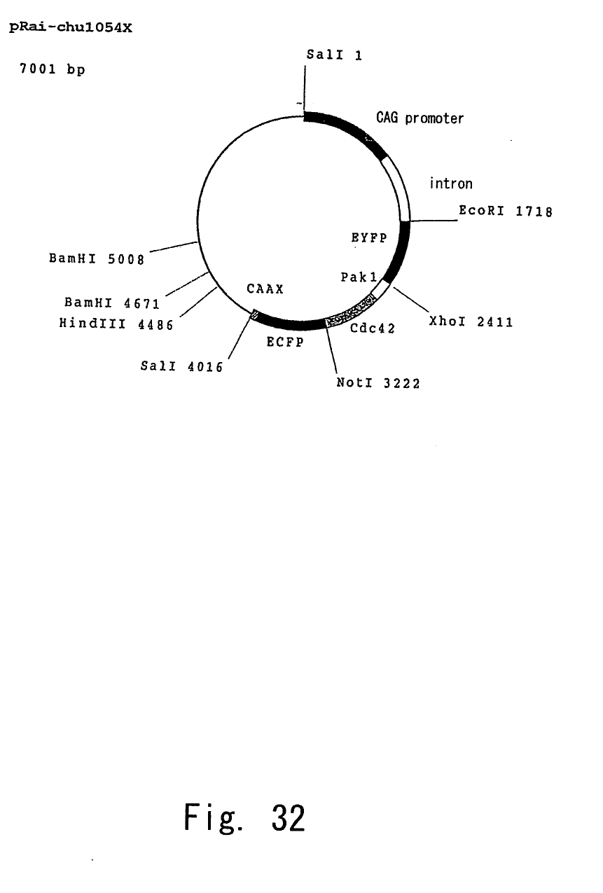

By using PCR-mediated method described in the examples 1 and 9 with cDNAs of Cdc42 (Genbank/EMBL accession number M57298) and Pak1 (Genbank/EMBL accession number NM002576) as templates, pRai-chu1054X was obtained. The structure (FIG. 32 and the nucleotide sequence of the coding region (sequence number: 39 and predicted amino acid sequence (sequence number: 40) are shown.

Detailed explanation is as follows.

| | | |
|---|---|---|
| nt | 1-684: | Aequorea EYFP |
| nt | 685-690: | Linker |
| nt | 691-939: | Pak1 |
| nt | 940-969: | Linker |
| nt | 970-1494: | Cdc42 |
| nt | 1495-1503: | Linker |
| nt | 1504-2214: | Aequorea ECFP |
| nt | 2215-2226: | Linker |
| nt | 2227-2286: | Carboxyl-terminal region of K-Ras (CAAX box) |

(2) Construction of Mutants of the Chimeric Gene

By the PCR-mediated mutagenesis, nucleotide thymine was substituted for guanine at position 1001 of pRai-chu1054X (sequence number 39), by which Val was substituted for Gly in the predicted amino acid sequence, generating pRai-chu1052X. In the mutant protein Rai-chu1052X, the GTPase activity is decreased, rendering this mutant constitutively active.

(3) Expression of Cdc42 Monitoring Protein Rai-chulO54X in Mammalian Cells and Their Analysis by Spectrometer.

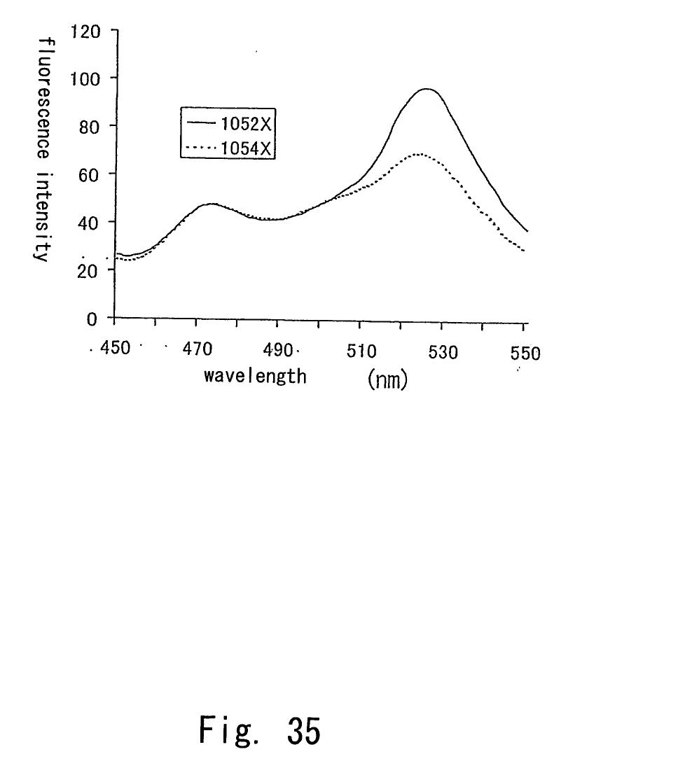

Rai-chu1054X and Rai-chu1052X were expressed in the cells and analyzed by the method described in the example 1-(4). The obtained spectral profiles are shown in FIG. 35. The FRET efficiency of the wild-type Rai-chulO54X is lower than that of the active form Rai-chu1052X.

### Example 14

### Construction of a Monitoring Protein for RhoA, Raichu-1214X

(1) Construction of a Chimeric Gene Between RhoA and mDia1.

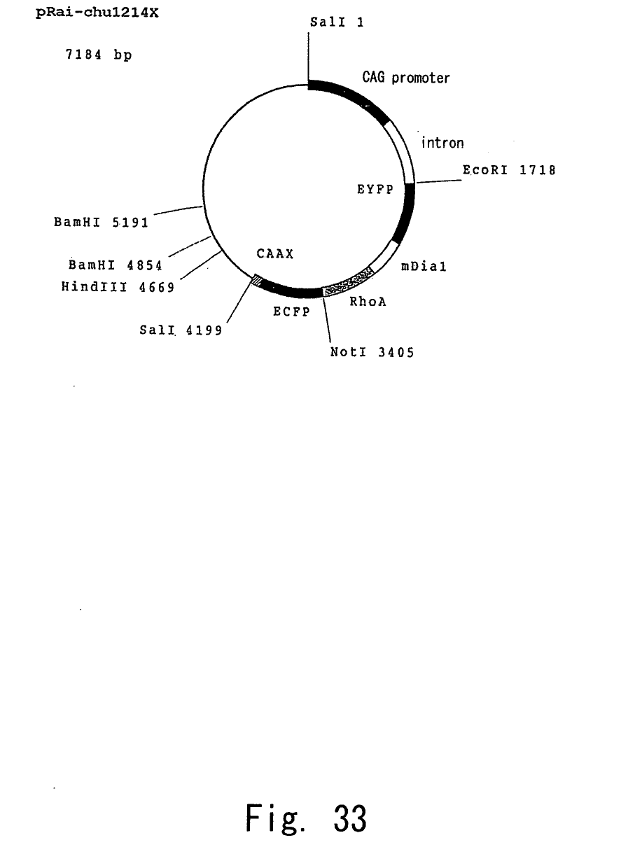

By using PCR method described in the examples 1 and 9, pRai-chu1214×was obtained with cDNAs of RhoA (Genbank/EMBL accession number L25080) and mDia1 (Genbank/EMBL accession number E17361) used as templates. The structure (FIG. 33 and the nucleotide sequence of the coding region (sequence number: 41 and predicted amino acid sequence (sequence number: 42) are shown.

Detailed explanation is as follows.

| | | |
|---|---|---|
| nt | 1-684: | Aequorea EYFP |
| nt | 685-696: | Linker |
| nt | 697-1092: | mDia1 |
| nt | 1093-1110: | Linker |
| nt | 1111-1677: | RhoA |
| nt | 1678-1686: | Linker |
| nt | 1687-2397: | Aequorea ECFP |
| nt | 2398-2409: | Linker |
| nt | 2410-2469: | Carboxyl-terminal region of K-Ras (CAAX box) |

(2) Construction of Mutants of the Chimeric Gene

By the PCR-mediated mutagenesis, nucleotide thymine and cytosine were substituted for adenine and guanine at positiosns 1298 and 1299 of pRai-chu1214×(sequence number 41), by which Leu was substituted for Gln in the predicted amino acid sequence, generating pRai-chu1220X. In the mutant protein Rai-chu1220X, the GTPase activity is decreased, rendering this mutant constitutively active.

(3) Expression of RhoA Monitoring Protein Rai-chu1054X in Mammalian Cells and Their Analysis by Spectrometer.

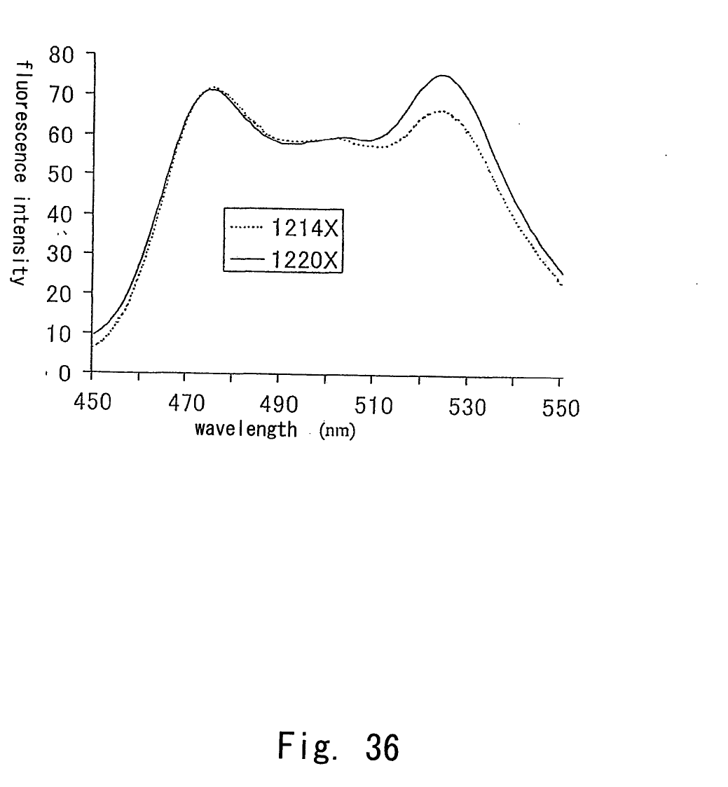

Rai-chu1214X and Rai-chu1220X were expressed in the cells and analyzed by the method described in the example 1 (4). The obtained profiles of spectrum are shown in FIG. 36. The FRET efficiency of the wild-type Rai-chu1214×is lower than that of the active form Rai-chu1220X.

### Example 15

### Visualization of Rac1 Activation in the COS1 Cells Expressing Rai-chu1011X.

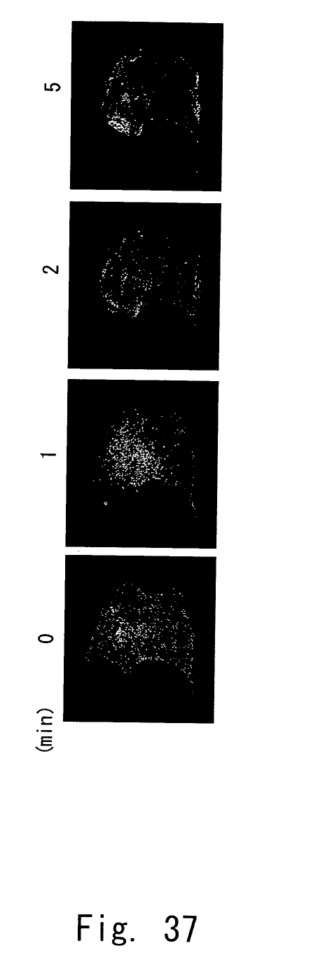

COS1 cells were replated to glass-base dishes. pRai-chu1011X described in example 12 was transfected into COS1 cells. After 24 hours, cells were imaged by the fluorescent microscope system described in example 1-(5). The time-course of fluorescence intensities of ECFP and EYFP and fluorescence ratio (EYFP/ECFP) in the cells stimulated with nerve growth factor are shown in FIG. 37. Within one minute after EGF stimulation, Rac1 is activated diffusely in the cells and then the activation was localized to the membrane ruffles, where cell membrane moves dynamically. Thus, the invented monitoring proteins enables us to obtain the spatio-temporal information on the activity of Rho-family G proteins. The pattern of the Rac1 activation was different from those of Ras or Rap1, further supporting the specificity of the monitoring proteins.

Sequences:

Sequence number 1: Nucleotide sequence of a primer based on the XhoI restriction sequence and the nucleotide sequence of human H-Ras.

Sequence number 2: Nucleotide sequence of a primer based on the nucleotide sequences of human c-Raf1 and human H-Ras.

Sequence number 3: Nucleotide sequence of a primer based on the KpnI restriction sequence and the nucleotide sequence of human c-Raf1.

Sequence number 4: Nucleotide sequence of a primer based on the NotI restriction sequence and the nucleotide sequence of human c-Raf1.

Sequence number 5: Nucleotide sequence of a primer based on the 5′ sequence of the multiple cloning site of pBluescript-SKII (+).

Sequence number 6: Nucleotide sequence of a primer based on the 3′ sequence of the multiple cloning site of pBluescript-SKII (+).

Sequence number 7: Nucleotide sequence of a primer based on the BamHI restriction sequence and the nucleotide sequence of EYFP.

Sequence number 8: Nucleotide sequence of a primer based on the restriction sequences of BamHI, KpnI, and XhoI and the nucleotide sequence of ECFP.

Sequence number 9: Nucleotide sequence of a primer based on the NotI restriction sequence and the nucleotide sequence of ECFP.

Sequence number 10: Nucleotide sequence of a primer based on the BglII restriction sequence and the nucleotide sequence of ECFP.

Sequence number 11: Nucleotide sequence of a plasmid based on the nucleotide sequences of human H-Ras, human c-Raf1, EYFP, and ECFP.

Sequence number 12: Amino acid sequence predicted from the nucleotide sequence of the plasmid shown in the sequence number 11.

Sequence number 13: Nucleotide sequence of a primer based on the XhoI restriction sequence and the nucleotide sequence of human Rap1A.

Sequence number 14: Nucleotide sequence of a primer based on the nucleotide sequences of human human RalGDS and human Rap1A.

Sequence number 15: Nucleotide sequence of a primer based on the nucleotide sequence of human RalGDS.

Sequence number 16: Nucleotide sequence of a primer based on the NotI restriction sequence and the nucleotide sequence of human RalGDS.

Sequence number 17: Nucleotide sequence of a primer based on the restriction sequences of BamHI, KpnI, and XhoI and the nucleotide sequence of ECFP.

Sequence number 18: Nucleotide sequence of a plasmid based on the nucleotide sequences of human Rap1A, human RalGDS, EYFP, and ECFP.

Sequence number 19: Amino acid sequence predicted from the nucleotide sequence of the plasmid shown in sequence number 18.

Sequence number 20: Nucleotide sequence of a primer based on the XhoI restriction sequence and the nucleotide sequence of human R-Ras.

Sequence number 21: Nucleotide sequence of a primer based on the KpnI restriction sequence and the nucleotide sequence of human R-Ras.

Sequence number 22: Nucleotide sequence of a plasmid based on the nucleotide sequences of human R-Ras, human c-Raf1, EYFP, and ECFP.

Sequence number 23: Amino acid sequence predicted from the nucleotide sequence of the plasmid shown in sequence number 22.

Sequence number 24: Nucleotide sequence of a primer based on the nucleotide sequence of human H-Ras.

Sequence number 25: Nucleotide sequence of a primer based on the nucleotide sequence of human H-Ras.

Sequence number 26: Nucleotide sequence of a plasmid based on the nucleotide sequences of human H-Ras, human c-Rafl, EYFP, and ECFP.

Sequence number 27: Amino acid sequence predicted from the nucleotide sequence of the plasmid shown in sequence number 29.

Sequence number 28: Nucleotide sequence of a primer based on the H-Ras binding region of human c-Raf1.

Sequence number 29: Nucleotide sequence of a primer based on the nucleotide sequence of ECFP.

Sequence number 30: Nucleotide sequence of a plasmid based on the nucleotide sequences of human Rap1A, human c-Raf1, EYFP, and ECFP.

Sequence number 31: Amino acid sequence predicted from the nucleotide sequence of the plasmid shown in sequence number 30.

Sequence number 32: Nucleotide sequence of a primer based on the nucleotide sequence of ECFP.

Sequence number 33: Nucleotide sequence of a plasmid based on the nucleotide sequences of human H-Ras, human c-Raf1, EYFP, ECFP, and human K-Ras.

Sequence number 34: Amino acid sequence predicted from the nucleotide sequence of the plasmid shown in sequence number 33.

Sequence number 35: Nucleotide sequence of a plasmid based on the nucleotide sequences of human Rap1A, human c-Raf1, EYFP, ECFP, and human K-Ras.

Sequence number 36: Amino acid sequence predicted from the nucleotide sequence of the plasmid shown in sequence number 33.

Sequence number 37: Nucleotide sequence of a plasmid based on the nucleotide sequences of human Rac1, human Pak1, EYFP, ECFP, and human K-Ras.

Sequence number 38: Amino acid sequence predicted from the nucleotide sequence of the plasmid shown in sequence number 37.

Sequence number 39: Nucleotide sequence of a plasmid based on the nucleotide sequences of human Cdc42, human Pak1, EYFP, ECFP, and human K-Ras.

Sequence number 40: Amino acid sequence predicted from the nucleotide sequence of the plasmid shown in sequence number 39.

Sequence number 41: Nucleotide sequence of a plasmid based on the nucleotide sequences of human RhoA, human mDia1, EYFP, ECFP, and human K-Ras.

Sequence number 42: Amino acid sequence predicted from the nucleotide sequence of the plasmid shown in sequence number 41.

The Value of this Invention for the Industry

The present invention provides monitoring proteins for the activity of low-molecular-weight GTP-binding proteins, cells and transgenic animals expressing the monitoring proteins useful for the measurement of the activity of low-molecular-weight GTP-binding proteins in non-destructive manners, methods for measurement of the activity of low-molecular-weight GTP-binding proteins which use the proteins, more in detail, methods that measure the ratio of GTP-bound to GDP-bound forms of the low-molecular weight GTP-binding proteins that are applicable with living cells, and screening procedures for the regulatory substances of low-molecular-weight GTP-binding proteins.

What is claimed is:

1. Monitoring proteins for low-molecular-weight GTP-binding proteins consisting of: fused proteins, wherein the fused proteins include at least said low-molecular-weight GTP-binding protein, a target protein of said low-molecular-weight GTP-binding proteins, a GFP donor protein, and a GFP acceptor protein, whole or part of which are directly or indirectly connected each other, in a state wherein each of the protein retains its function.

2. Monitoring proteins for low-molecular-weight GTP-binding proteins according to claim 1, wherein subcellular localization signals are further connected.

3. Monitoring proteins for low-molecular-weight GTP-binding proteins according to claims 1 and 2, wherein spacer peptides are intercalated between the low-molecular-weight GTP-binding protein and the target protein.

4. Monitoring proteins for low-molecular-weight GTP-binding proteins according to claims 1 to 3, wherein the low-molecular-weight GTP-binding protein belongs to Ras-superfamily GTP-binding proteins.

5. Monitoring proteins for low-molecular-weight GTP-binding proteins according to claims 1 to 4, wherein the low-molecular-weight GTP-binding protein belongs to Ras-family GTP-binding proteins.

6. Monitoring proteins for low-molecular-weight GTP-binding proteins according to claims 1 to 5, wherein the low-molecular-weight GTP-binding protein is selected from H-Ras, K-Ras, N-Ras, R-Ras, Rap1A, Rap1B, Rap2A, and Rap2B.

7. Monitoring proteins for low-molecular-weight GTP-binding proteins according to claims 1 to 4, wherein the low-molecular-weight GTP-binding protein belongs to Rho-family GTP-binding proteins.

8. Monitoring proteins for low-molecular-weight GTP-binding proteins according to claims 1 to 4 or 7, wherein the low-molecular-weight GTP-binding protein is selected from RhoA, RhoB, RhoC, Rac1, Rac2, and Cdc42.

9. Monitoring proteins for low-molecular-weight GTP-binding proteins according to claims 1 to 8, wherein the target proteins is selected from Raf, RalGDS, Pak, mDia1.

10. Monitoring proteins for low-molecular-weight GTP-binding proteins according to claims 1 to 9, wherein the GFP acceptor protein is one of EGFP and EYFP.

11. Monitoring proteins for low-molecular-weight GTP-binding proteins according to claims 1 to 10, wherein the GFP donor protein is one of ECFP and EBFP.

12. Monitoring proteins for low-molecular-weight GTP-binding proteins according to claims 1 to 11, wherein at least one of the low-molecular-weight GTP-binding protein and the target protein contains mutation.

13. Monitoring proteins for low-molecular-weight GTP-binding proteins according to claims 1 to 12, wherein the amino-terminus and/or carboxyl-terminus of the low-molecular-weight GTP-binding protein is deleted by at least one amino acid.

14. Monitoring proteins for low-molecular-weight GTP-binding proteins according to claims 1 to 13, wherein at least one of the amino-terminus and carboxyl-terminus of the GFP donor protein is deleted by at least one amino acid.

15. Monitoring proteins for low-molecular-weight GTP-binding proteins according to claims 1 to 6 and 9 to 14, wherein the low-molecular-weight GTP-binding protein is H-Ras and the target protein is Raf.

16. Monitoring proteins for low-molecular-weight GTP-binding proteins according to claims 1 to 6 and 9 to 14, wherein the low-molecular-weight GTP-binding protein is Rap1A and the target protein is RalGDS.

17. Monitoring proteins for low-molecular-weight GTP-binding proteins according to claims 1 to 4 and 7 to 14, wherein the low-molecular-weight GTP-binding protein is Rac1 and the target protein is Pak.

18. Monitoring proteins for low-molecular-weight GTP-binding proteins according to claims 1 to 4 and 7 to 14, wherein the low-molecular-weight GTP-binding protein is Cdc42 and the target protein is Pak.

19. Monitoring proteins for low-molecular-weight GTP-binding proteins according to claims 1 to 4 and 7 to 14, wherein the low-molecular-weight GTP-binding protein is RhoA and the target protein is mDia.

20. Monitoring proteins for low-molecular-weight GTP-binding proteins according to claims 1 to 6 and 9 to 15, wherein the low-molecular-weight GTP-binding protein is H-Ras, the target protein is Raf, GFP donor protein is ECFP, and GFP acceptor protein is EYFP.

21. Monitoring proteins for low-molecular-weight GTP-binding proteins according to claim 20, wherein, from the amino-terminus, EYFP, H-Ras, Raf, and ECFP are ligated directly or indirectly.

22. Monitoring proteins for low-molecular-weight GTP-binding proteins according to claims 1 to 6, 9, 14, and 16, wherein the low-molecular-weight GTP-binding protein is Rap1A, the target protein is RalGDS, GFP donor protein is ECFP, and GFP acceptor protein is EYFP.

23. Monitoring proteins for low-molecular-weight GTP-binding proteins according to claim 22, wherein, from the amino-terminus, EYFP, Rap1A, RalGDS, and ECFP are ligated directly or indirectly.

24. Monitoring proteins for low-molecular-weight GTP-binding proteins according to claims 1 to 4, 7 to 14, and 17, wherein the low-molecular-weight GTP-binding protein is Rac1, the target protein is Pak, GFP donor protein is ECFP, and GFP acceptor protein is EYFP.

25. Monitoring proteins for low-molecular-weight GTP-binding proteins according to claim 24, wherein, from the amino-terminus, EYFP, Pak, Rac1, and ECFP are ligated directly or indirectly.

26. Monitoring proteins for low-molecular-weight GTP-binding proteins according to claims 1 to 4, 7 to 14, and 18, wherein the low-molecular-weight GTP-binding protein is Cdc42, the target protein is Pak, GFP donor protein is ECFP, and GFP acceptor protein is EYFP.

27. Monitoring proteins for low-molecular-weight GTP-binding proteins according to claim 26, wherein, from the amino-terminus, EYFP, Pak, Cdc42, and ECFP are ligated directly or indirectly.

28. Monitoring proteins for low-molecular-weight GTP-binding proteins according to claims 1 to 4, 7 to 14, and 19, wherein the low-molecular-weight GTP-binding protein is RhoA, the target protein is mDia, GFP donor protein is ECFP, and GFP acceptor protein is EYFP.

29. Monitoring proteins for low-molecular-weight GTP-binding proteins according to claim 28, wherein, from the amino-terminus, EYFP, mDia, RhoA, and ECFP are ligated directly or indirectly.

30. Genes which encode monitoring proteins for low-molecular-weight GTP-binding proteins according to claims 1 to 29.

31. Expression vectors comprising:
the genes according to claim 30.

32. Expression vectors according to claim 30, wherein the expression vectors are expression plasmids.

33. Cells transformed by the expression vectors according to claims 31 and 32.

34. Transgenic animals comprising:
expression vectors according to claims 31 and 32.

35. A method for measuring the activity of the low-molecular-weight GTP-binding proteins comprising: the step of detecting FRET of the monitoring proteins for low-molecular-weight GTP-binding proteins according to claims 1 to 29.

36. A method for measuring the activity of the low-molecular-weight GTP-binding proteins comprising: the step of detecting FRET of the monitoring proteins for the low-molecular-weight GTP-binding proteins in transgenic animals according to claim 34 or cells according to claim 33.

37. A method for measuring the activity of the low-molecular-weight GTP-binding proteins according to claim 36 further comprising: the step of calculating molar ratio of GTP/GDP on said protein by measuring the amounts of GTP-bound low-molecular-weight GTP-binding protein and the low-molecular-weight GTP-binding protein bound to GDP which is generated upon the release of inorganic phosphate from GTP.

38. A screening method for the regulator of the activity of low-molecular-weight GTP-binding proteins comprising: (a) the step of culturing cells according to claim 33 in the presence of the specimens and (b) the step of measuring the activity change of low-molecular-weight GTP-binding proteins.
